SEQUENCE LISTING (120 sequences)

SEQ ID NO: 1 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 2 - protein, 131 aa, artificial sequence
  Pro to Thr at position 5 of bacteriophage AP205
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 3 - DNA, 3635 bp, artificial sequence
  expressing AP205 coat protein
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 4 - DNA, 39 bp, artificial sequence
  to clone GSGG at the N-of AP205 coat protein
tgccatgggatccggaggggcaaataagccaatgcaacc

SEQ ID NO: 5 - DNA, 31 bp, artificial sequence
  to clone GSGG to the N terminus of AP205 coat protein
nnaagcttaagcagtagtatcagacgatacg

SEQ ID NO: 6 - DNA, 60 bp, artificial sequence
  to clone GSGTAGGGSGS to the N- of AP205 coat protein using p378-2 as template
tgccatgggttccggaaccgcgggcgggggatccggttcggcaaataagccaatgcaacc

SEQ ID NO: 7 - DNA, 30 bp, artificial sequence
  to clone GSGG to the C- of AP205 coat protein using p283-58 as template
nntctagaattttctgcgcacccatcccgg

SEQ ID NO: 8 - DNA, 40 bp, artificial sequence
  to clone GSGG to the C- of AP205 coat protein using p283-58 as template
tgatgcatcctccggatccagcagtagtatcagacgatac

SEQ ID NO: 9 - DNA, 46 bp, artificial sequence
  to clone  GTAGGGSG  to the C- of AP205 coat protein using p409-44 as template
tgatgcataatccggaaccgcctcctgcggttccagcagtagtatc

SEQ ID NO: 10 - protein, 15 aa, artificial sequence
  D2 peptide
TSNGSNPSTSYGFAN

SEQ ID NO: 11 - DNA, 63 bp, artificial sequence
  to anneal with oligo2197 to create D2 peptide
cctccggaacttccaacggaagcaatccgagcacttcgtacggtttcgcgaattaatgcatcg

SEQ ID NO: 12 - DNA, 63 bp, artificial sequence
  to anneal with oligo2.196 to create D2 peptide
cgatgcattaattcgcgaaaccgtacgaagtgctcggattgcttccgttggaagttccggagg

SEQ ID NO: 13 - DNA, 66 bp, artificial sequence
  to anneal with oligo590 to create De peptide at the N- of AP205 coat protein
ctccatgggaacttccaacggaagcaatccgagcacttcgtacggtttcgcgaatggatccggatc

SEQ ID NO: 14 - DNA, 66 bp, artificial sequence
  to anneal with oligo 590 to create D2 peptide at the N- of AP205 coat protein
gatccggatccattcgcgaaaccgtacgaagtgctcggattgcttccgttggaagttcccatggag

SEQ ID NO: 15 - protein, 8 aa, artificial sequence
  Angiotensin I fragment
DRVYIHPF

SEQ ID NO: 16 - DNA, 42 bp, artificial sequence
  to anneal with oligo3.217 to create angio I fragment at the C- of AP205 coat protein
gatccggagatcgtgtatacatccatccattctaatgcattg

SEQ ID NO: 17 - DNA, 42 bp, artificial sequence
  to anneal with oligo 3.216 to create angio I fragment at the C- of AP205 coat protein
caatgcattagaatggatggatgtatacacgatctccggatc

SEQ ID NO: 18 - DNA, 45 bp, Artificial Sequence
  to anneal with oligo 3.219 to create angio I fragment at the N- of AP205 coat protein
tcccatgggagatcgtgtatacatccatccattcggatccggaac

SEQ ID NO: 19 - DNA, 45 bp, artificial sequence
  to anneal with oligo 3.218 to create angio I fragment at the N-of AP205 coat protein
gttccggatccgaatggatggatgtatacacgatctcccatggga

SEQ ID NO: 20 - protein, 10 aa, Homo sapiens
EHWSYGLRPG

SEQ ID NO: 21 - DNA, 48 bp, artificial sequence
  to anneal with oligo 4.57 to create GnRH at the C- of AP205 coat protein
gttccggagaacactggtcctatggactcaggcctggttaatgcattg

SEQ ID NO: 22 - DNA, 48 bp, artificial sequence
  to anneal with oligo 4.56 to create GnRH at the C- of AP205 coat protein
caatgcattaaccaggcctgagtccataggaccagtgttctccggaac

SEQ ID NO: 23 - protein, 55 aa, Human immunodeficiency virus
GVGFPVRPQVPLRPMTYKAAVDLSHFLKEKGGLEGPGIRYPLTFGWCFKLVPVEP

SEQ ID NO: 24 - DNA, 28 bp, artificial sequence
  to anneal with 3.222 to create the 34 aa of Nef 54 at the C-of AP205 coat protein
gatccggaggtgtgggtttcccggttcg

SEQ ID NO: 25 - DNA, 38 bp, artificial sequence
  to anneal with 3.242 to create the 34 aa of Nef 56 at the C- of AP205 coat protein
gaaaagggtggcctggaaggtccaggtatccgttatcc

SEQ ID NO: 26 - DNA, 38 bp, artificial sequence
  to anneal with oligo 3.225 to creat the 21aa of Nef 56 to the C- of AP205 coat protein
ggataacggatacctggaccttccaggccacccttttc

SEQ ID NO: 27 - DNA, 25 bp, artificial sequence
  to anneal with 3.223 to create 21 aa of Nef 56 to the C- of AP205 coat protein
caatgcattacggttcaaccggcac

SEQ ID NO: 28 - DNA, 44 bp, artificial sequence
  TGA creation for mosaic V LP
gtatcgtctgatactactgcttgaggaaccgcaggaggcggttc

SEQ ID NO: 29 - DNA, 44 bp, artificial sequence
  to create TGA for mosaic VLP
gaaccgcctcctgcggttcctcaagcagtagtatcagacgatac

SEQ ID NO: 30 - DNA, 28 bp, artificial sequence
  Nco I site
nnccatggcaaataagccaatgcaaccg

SEQ ID NO: 31 - DNA, 19 bp, artificial sequence
  complementary to the non coding region 23 nucleotides downstream of the C-terminus of Nef56
gatttaggtgacactatag

SEQ ID NO: 32 - protein, 14 aa, artificial sequence
  p33 peptide
AKSLKAVYNFATMA

SEQ ID NO: 33 - DNA, 57 bp, artificial sequence
  to anneal with 3.310 to create p33 peptide at the N- of AP205 coat protein
ggccatggctaagtccctgaaggctgtatataacttcgcgactatggcatccggagg

SEQ ID NO: 34 - DNA, 57 bp, artificial sequence
  to anneal with 3.309 to create p33 peptide at the N-of AP205 coat protein
cctccggatgccatagtcgcgaagttatatacagccttcagggacttagccatggcc

SEQ ID NO: 35 - protein, 4 aa, artificial sequence
  N- short spacer
GSGG

SEQ ID NO: 36 - protein, 11 aa, artificial sequence
  N- long spacer
GSGTAGGGSGS

SEQ ID NO: 37 - protein, 3 aa, artificial sequence
  C-short spacer
GSG

SEQ ID NO: 38 - protein, 8 aa, artificial sequence
  C- long spacer
GTAGGGSG

SEQ ID NO: 39 - DNA, 42 bp, artificial sequence
  primer for cloning p33 into fr
cgaaatctcttaaagcggtttacaacttcgctaccatggctt

SEQ ID NO: 40 - DNA, 42 bp, artificial sequence
  primer to clone p33 to fr
cgaagccatggtagcgaagttgtaaaccgctttaagagattt

SEQ ID NO: 41 - protein, 20 aa, Mus musculus
SSQNSSDKPVAHVVANHQVE

SEQ ID NO: 42 - protein, 131 aa, artificial sequence
  a mutein of the coat protein of AP205
MANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 43 - protein, 24 aa, artificial sequence
  M2 sequence of Influnza
SLLTEVETPIRNEWGCRCNDSSDG

SEQ ID NO: 44 - protein, 24 aa, artificial sequence
  M2 pr8 for mouse
SLLTEVETPIRNEWGCRCNGSSDG

SEQ ID NO: 45 - protein, 31 aa, artificial sequence
  CCR5 PNt domain
MDYQVSSPIYDINYYTSEPCQKINVKQIAAR

SEQ ID NO: 46 - protein, 10 aa, artificial sequence
  CCR5 ECL2A
RSQKEGLHYT

SEQ ID NO: 47 - protein, 17 aa, Homo sapiens
EGPWLEEEEEAYGWMDF

SEQ ID NO: 48 - protein, 39 aa, artificial sequence
  CXCR4 1-39
MEGISIYTSDNYTEEMGSGDYDSMKEPCFREENANFNKI

SEQ ID NO: 49 - protein, 16 aa, artificial sequence
  CXCR4 176-185
SEQIDENVSEADDRYI

SEQ ID NO: 50 - protein, 20 aa, artificial sequence
  artificial sequence
CVVASQLRANISHKDMQLGR

SEQ ID NO: 51 - protein, 16 aa, artificial sequence
  CETP 461-479
FGFPEHLLVDFLQSLS

SEQ ID NO: 52 - protein, 10 aa, homo sapiens
KRPPGFSPFR

SEQ ID NO: 53 - protein, 9 aa, homo sapiens
KRPPGFSPF

SEQ ID NO: 54 - protein, 28 aa, Homo sapiens
GSSFLSPEHQRVQQRKESKKPPAKLQPR

SEQ ID NO: 55 - protein, 8 aa, artificial sequence
  ghrelin 1-8
GSSFLSPE

SEQ ID NO: 56 - protein, 10 aa, artificial sequence
  ghrelin 1-10
GSSFLSPEHQ

SEQ ID NO: 57 - protein, 12 aa, artificial sequence
  ghrelin 1-12
GSSFLSPEHQRV

SEQ ID NO: 58 - protein, 13 aa, artificial sequence
  dog ghrelin 1-13
GSSFLSPEHQKLQ

SEQ ID NO: 59 - protein, 13 aa, artificial sequence
  cat ghrelin 1-13
GSSFLSPEHQKVQ

SEQ ID NO: 60 - protein, 34 aa, homo sapiens
ELGPQGPPHLVADPSKKQGPWLEEEEEAYGWMDF

SEQ ID NO: 61 - protein, 9 aa, artificial sequence
  gastrin 1-9
EGPWLEEEE

SEQ ID NO: 62 - protein, 27 aa, artificial sequence
  fragment 21-47 of PreS1
PLGFFPDHQLDPAFRANTANPDWDFNP

SEQ ID NO: 63 - DNA, 99 bp, artificial sequence
  primer for clone PreS1 at the C-terminus
cctccggaccgctgggcttcttcccggatcaccagctggatccggccttccgcgccaacaccgcgaacccggattgggatttcaacccgtaatgcatcg

SEQ ID NO: 64 - DNA, 99 bp, artificial sequence
  primer for clone PreS1 at the C-terminus
cgatgcattacgggttgaaatcccaatccgggttcgcggtgttggcgcggaaggccggatccagctggtgatccgggaagaagcccagcggtccggagg

SEQ ID NO: 65 - DNA, 102 bp, artificial sequence
  primer for clone PreS1 at the N-terminus
ctccatgggaccgctgggcttcttcccggatcaccagctggatccggccttccgcgccaacaccgcgaacccggattgggatttcaacccgggatccggatc

SEQ ID NO: 66 - DNA, 102 bp, artificial sequence
  primer for clone PreS1 at the N-terminus
gatccggatcccgggttgaaatcccaatccgggttcgcggtgttggcgcggaaggccggatccagctggtgatccgggaagaagcccagcggtcccatggag

SEQ ID NO: 67 - protein, 130 aa, bacteriophage AP205
ANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 68 - protein, 130 aa, artificial sequence
  de Met of SEQ ID NO2 of bacteriophage AP205
ANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 69 - protein, 130 aa, artificial sequence
  de Met of SEQ ID NO42 of bacteriophage AP205
ANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 70 - DNA, 10 bp, artificial sequence
  unmethylated CpG palindromic sequence
gacgatcgtc

SEQ ID NO: 71 - DNA, 30 bp, artificial sequence
  G10
gggggggggggacgatcgtcgggggggggg

SEQ ID NO: 72 - protein, 31 aa, artificial sequence
  artificial sequence
RSQKEGLHYTSSSHFPYSQYQFWKNFQTLKI

SEQ ID NO: 73 - protein, 33 aa, artificial sequence
  CCR5 ECL2A cylic sequence with C to S at position 20
CRSQKEGLHYTSSSHFPYSQYQFWKNFQTLKIC

SEQ ID NO: 74 - protein, 12 aa, artificial sequence
  CCR5 ECL2A cyclic
CRSQKEGLHYTC

SEQ ID NO: 75 - protein, 39 aa, artificial sequence
  CXCR4 Nt cyclic Cys 28 to Ser
MEGISIYTSDNYTEEMGSGDYDSMKEPSFREENANFNKI

SEQ ID NO: 76 - DNA, 119 bp, artificial sequence
  primer 1 for annealling CXCR4 Nt cyclic
catggaaggaatttccatatatacttcggacaactacaccgaggaaatgggtagcggcgactacgacagcatgaaagaaccatccttccgcgaggagaatgcaaattttaataaaattt

SEQ ID NO: 77 - DNA, 119 bp, artificial  sequence
  primer 2 for anealling CXCR4 Nt cyclic
ccggaaattttattaaaatttgcattctcctcgcggaaggatggttctttcatgctgtcgtagtcgccgctacccatttcctcggtgtagttgtccgaagtatatatggaaattccttc

SEQ ID NO: 78 - protein, 10 aa, Artificial Sequence
  P33 fragment 2
LKAVYNFATM

SEQ ID NO: 79 - DNA, 48 bp, artificial sequence
  primer 1 for annealing P33 fragment 2
cctccggactgaaagctgtgtataacttcgcgactatgtaatgcatcg

SEQ ID NO: 80 - DNA, 48 bp, artificial sequence
  primer 2 for p33 fragment 2
cgatgcattacatagtcgcgaagttatacacagctttcagtccggagg

SEQ ID NO: 81 - DNA, 42 bp, artificial sequence
  primer 1 for ghrelin
gttccggagggagctccttcctgtctccggaataatgcatgt

SEQ ID NO: 82 - DNA, 42 bp, artificial sequence
  primer 2 for Ghrelin
acatgcattattccggagacaggaaggagctccctccggaac

SEQ ID NO: 83 - protein, 26 aa, artificial sequence
  M2 with MG added at the N-terminus
MGSLLTEVETPIRNEWGCRCNDSSDG

SEQ ID NO: 84 - DNA, 90 bp, artificial sequence
  primer 1 for M2 with MG added at the N-terminus
ggccatgggatctctgctgaccgaagttgaaaccccgattcgtaatgaatggggttgccgttgcaatgattcttctgatggttccggagg

SEQ ID NO: 85 - DNA, 91 bp, artificial sequence
  primer 2 for M2 with MG added at the N-terminus
cctccggaaccatcagaagaatcattgcaacggcaaccccattcattacgaatcggggtttcaacttcggtcagcagagatcccatggcca

SEQ ID NO: 86 - DNA, 111 bp, artificial sequence
  primer 1 for cECL2c
ccggatgtcgatcgcagaaggaaggcctacattacacatcctcatctcacttcccatattctcaatatcaattctggaagaatttccaaactctgaagatctgttaatgca

SEQ ID NO: 87 - DNA, 103 bp, artificial sequence
  primer 2 for cECL2c
ttaacagatcttcagagtttggaaattcttccagaattgatattgagaatatgggaagtgagatgaggatgtgtaatgtaggccttccttctgcgatcgacat

SEQ ID NO: 88 - DNA, 54 bp, artificial sequence
  primer 1 for cECL2ac
gttccggatgtcgatcgcagaaggaaggcctacattacacatgctaatgcatgt

SEQ ID NO: 89 - DNA, 54 bp, artificial sequence
  primer 2 for cECL2ac
acatgcattagcatgtgtaatgtaggccttccttctgcgatcgacatccggaac

SEQ ID NO: 90 - protein, 31 aa, artificial sequence
  CCR5 Nt with Cysteine 20 changed to S
MDYQVSSPIYDINYYTSEPSQKINVKQIAAR

SEQ ID NO: 91 - protein, 31 aa, artificial sequence
  CCR5 ECL2
RSQKEGLHYTCSSHFPYSQYQFWKNFQTLKI

SEQ ID NO: 92 - DNA, 95 bp, artificial sequence
  primer 1 for CCR5 PNt
catggattatcaagtctcgagccctatctatgacattaactattacacttcggaaccttcgcagaagattaacgttaaacaaattgcagcacgtt

SEQ ID NO: 93 - DNA, 95 bp, artificial sequence
  primer 2 for CCR5 PNt
ccggaacgtgctgcaatttgtttaacgttaatcttctgcgaaggttccgaagtgtaatagttaatgtcatagatagggctcgagacttgataatc

SEQ ID NO: 94 - DNA, 21 bp, artificial sequence
  1668Pt
tccatgacgttcctgaataat

SEQ ID NO: 95 - DNA, 20 bp, artificial sequence
  NK pt
ggggtcaacgttgagggggg

SEQ ID NO: 96 - protein, 52 aa, artificial sequence
  M2 peptide dimers with GSSG linker
SLLTEVETPIRNEWGCRCNDSSDGGSSGSLLTEVETPIRNEWGCRCNDSSDG

SEQ ID NO: 97 - protein, 85 aa, artificial sequence
  M2 peptide trimers with GSSG linker
APGSGSLLTEVETPIRNEWGCRCNDSSDGGSSGSLLTEVETPIRNEWGCRCNDSSDGGSSGSLLTEVETPIRNEWGCRCNDSSDG

SEQ ID NO: 98 - protein, 16 aa, artificial sequence
  HIV env 1
SLEQIWNNMTWMQWDK

SEQ ID NO: 99 - protein, 16 aa, artificial sequence
  HIV env 2
SLEQIWNNMTWMQWDR

SEQ ID NO: 100 - protein, 12 aa, artificial sequence
  HIV env 3
IWNNMTWMQWDR

SEQ ID NO: 101 - protein, 7 aa, artificial sequence
  HIV env 4
WASLWNW

SEQ ID NO: 102 - protein, 10 aa, artificial sequence
  HIV env 5
NWFDISNWLW

SEQ ID NO: 103 - protein, 16 aa, artificial sequence
  HIV env 6
LLELDKWASLWNWFNL

SEQ ID NO: 104 - protein, 6 aa, artificial sequence
  HIV env 7
ELDKWA

SEQ ID NO: 105 - protein, 34 aa, artificial sequence
  HIV env 8
WMEWDREINNYTSLIHSLIEESQNQQEKNEQELL

SEQ ID NO: 106 - protein, 6 aa, artificial sequence
  HIV env 9
CSKLIC

SEQ ID NO: 107 - protein, 19 aa, artificial sequence
  HIV env 10
GFLGAAGSTMGAASITLVQ

SEQ ID NO: 108 - protein, 26 aa, artificial sequence
  HIV env 11
QQNNLLRAIEAQQHLLQLTVWGIKQL

SEQ ID NO: 109 - protein, 6 aa, artificial sequence
  HiV env 12
GIVQQQ

SEQ ID NO: 110 - protein, 25 aa, artificial sequence
  HIV env 13
QLLGIWGCSGKLICTTAVPWNSSWS

SEQ ID NO: 111 - protein, 14 aa, artificial sequence
  HIV env 14
NAKTIIVQLNQSVE

SEQ ID NO: 112 - protein, 17 aa, artificial sequence
  HIV env 15
GGNSNNESEIFRPGGGD

SEQ ID NO: 113 - protein, 35 aa, artificial sequence
  HIV env 16
VAPTKAKRRVVQREKRAVGIGALFLGFLGAAGSGC

SEQ ID NO: 114 - protein, 10 aa, homo sapiens
QHWSYGLRPG

SEQ ID NO: 115 - protein, 7 aa, Homo sapiens
DRVYIHP

SEQ ID NO: 116 - protein, 10 aa, Homo sapiens
DRVYIHPFHL

SEQ ID NO: 117 - protein, 6 aa, artificial sequence
  Ab 1-6
DAEFRH

SEQ ID NO: 118 - protein, 33 aa, artificial sequence
  CCR5 ECL2 cyclic
CRSQKEGLHYTCSSHFPYSQYQFWKNFQTLKIC

SEQ ID NO: 119 - protein, 48 aa, artificial sequence
  HIV gag
QGQMVHQAISPRTLNAWVKAFSPEVIPMFSALSEGATPQDLNTMLNTV

SEQ ID NO: 120 - protein, 49 aa, artificial sequence
  HIV gag with lys at the C terminus
QGQMVHQAISPRTLNAWVKAFSPEVIPMFSALSEGATPQDLNTMLNTVK

(19) United States
(12) Patent Application Publication
(10) Pub. No.: US 2008/0292652 A1
(43) Pub. Date: Nov. 27, 2008

(54) Virus-Like Particles Comprising a Fusion Protein of the Coat Protein of Ap205 and an Antigenic Polypeptide

(75) Inventors: Martin F Bachmann, Seuzach (CH); Alain Tissot, Zurich (CH); Gary Jennings, Zurich (CH); Regina Renhofa, Riga (LV); Paul Pumpens, Riga (LV); Indulis Cielens, Riga (LV)

Correspondence Address: STERNE, KESSLER, GOLDSTEIN & FOX P.L.L.C., 1100 NEW YORK AVENUE, N.W., WASHINGTON, DC, 20005, US

(73) Assignee: CYTOS BIOTECHNOLOGY AG, ZURICH-SCHLIEREN (CH)

(21) Appl. No.: 11/663,350

(22) PCT Filed: Sep. 21, 2005
(86) PCT No.: PCT/EP05/54721

§ 371 Date: Apr. 14, 2008

Related U.S. Application Data
(60) Provisional application No. 60/611,308, filed on Sep. 21, 2004.

(30) Foreign Application Priority Data
Jun. 14, 2005 (EP) 05105229.8

(51) Int. Cl. A61K 39/00; C12N 7/00; C07K 7/00; C12N 15/11; C12P 21/04; A61P 37/00

(52) U.S. Cl. 424/199.1; 435/235.1; 530/331; 530/330; 530/329; 530/328; 536/23.4; 435/69.7

(57) ABSTRACT

The present invention is in the fields of medicine, immunology, virology and molecular biology. The present invention provides a composition comprising a modified virus-like (VLP) particle derived from RNA bacteriophage AP205. The invention also provides a process for producing the aforementioned VLP. The modified VLP disclosed in the present invention is useful in the production of compositions for inducing immune responses for the prevention or treatment of diseases, disorders including infectious diseases, allergies, cancers and drug addiction. Moreover, the modified VLP disclosed in the present invention is, in particular, useful to efficiently induce self-specific immune responses, in particular antibody responses.

### BACKGROUND OF THE INVENTION

1. Field of the Invention

The present invention is in the fields of medicine, immunology, virology and molecular biology.

The present invention provides a composition comprising a modified virus-like (VLP) particle derived from RNA bacteriophage AP205. The invention also provides a process for producing the aforementioned VLP. The modified VLP disclosed in the present invention is useful in the production of compositions for inducing immune responses for the prevention or treatment of diseases, disorders including infectious diseases, allergies, cancers and drug addiction. Moreover, the modified VLP disclosed in the present invention is, in particular, useful to efficiently induce self-specific immune responses, in particular antibody responses.

2. Related Art

At least two conditions have to be met in order to induce an immune response towards foreign epitope, which is fused to the coat protein of a virus. First of all the fusion of a foreign sequence should not interfere with the assembly of the coat protein into a virus-like particle; secondly the foreign epitope should be displayed on the surface of the virus-like particle. The fusion of amino acid sequences to coat proteins of RNA phages has been described in the past. For example, insertion of epitopes into the AB loop of the coat protein of MS2 phage has been described (WO 92/13081; Mastico et al. J. Gen. Virol. (1993) 74:541-548). The N- and C-terminal fusion of epitopes have not been described for MS2 phage. A significant limitation of this technology is that through insertion into the AB loop of MS2, polypeptides may be forced into a conformation which differs from their native one.

Insertion of amino acid sequences between position 2 and 3, between position 50 and 52 (exposed on the inner surface of the fr capsid) or between amino acid 128 and 129 of the coat protein of RNA phage fr has also been reported (Pushko P. et al. Protein Eng (1993) 8: 883-891)). Pushko et al. also reported that alterations of the N-terminus of the Fr CP may affect the assembly at quasi-3-fold axes since several N-terminal insertion mutants demonstrate assembly to dimers only (Pushko, p. 890, last paragraph, Protein Eng, (1993) 8: 883-891). Insertion of three amino acid sequence before the last C-terminal residue of the fr coat protein allowed capsid assembly, whereas another longer epitope prevented capsid assembly. Accessibility of the three amino acid epitope insertion was not assessed. Thus only internal insertions of amino acids into the coat protein fr have been described to date.

In a number of instances, however, the presence of a free N- or a free C-terminus of the epitope is an important element for epitope recognition. For example, Seubert P. et al. (Neurobiol. (2004), Aging 25: S588) described that while mapping the Aβ epitope recognized by the antibodies elicited by respective vaccines, they found that in 41 of the mapped samples, the predominant antibody epitope was to the free amino-terminus of Aβ. Likewise, antibodies specific for the C-terminus of Angiotensin II have also been described (Budisavljevic M. et al. (1988) J. Immunol. 140:3059-3065).

Fusion of epitopes to the C-terminus has only been reported in the truncated form of the s A1 extension of RNA phage Qβ, which subsequently assembles only into a mosaic VLP. These are particles assembled from of a mixture of both A1 subunits displaying the epitope and wild type coat proteins devoid of it. No particles where obtained when only A1 extension displaying the epitope was expressed in E. coli (Vasiljeva et al. (1998) FEBS Letters 431: 7-11). These mosaic particles, however, display an epitope in a lower density, which might be problematic for its use as vaccines. One of the problems is that low density of antigen display may fail to induce sufficient immune response, in particular to break self-tolerance if the antigen is a self antigen (Bachmann & Zinkernagel, Immunol. Today 17:553-558 (1996)).

There is therefore a need in the field to identify coat proteins of viruses, to which a large variety of antigens may be fused, and wherein the resulting fusion proteins retain the capability of forming VLPs and displaying the antigens on the outer surface of the VLPs. Furthermore, there is a need to find coat proteins of viruses, which allow the fusion of foreign epitopes so that a free end of the epitopes may be accessible if that free end accounts for a strong immunogenicity.

### SUMMARY OF THE INVENTION

We have surprisingly found that a large variety of polypeptides can be fused to the N- or C-terminus of the coat protein of AP205 and the resulting fusion proteins form virus-like particles when expressed in a host, typically and preferably in E. coli. Furthermore we have surprisingly found that, if the polypeptide comprises at least one antigen, the antigen or at least one antigenic site of the antigen is displayed on the outer surface of the assembled VLPs.

Thus, in the first aspect, the invention provides a modified virus-like particle (VLP) comprising at least one fusion protein, wherein said at least one fusion protein comprises: (a) a first polypeptide; and (b) a second polypeptide; and wherein the first polypeptide is a coat protein, or a mutein thereof, of AP205 bacteriophage, and wherein said second polypeptide is fused to said first polypeptide either at the N- or at the C-terminus of the first polypeptide.

In one preferred embodiment, the second polypeptide comprises at least one antigen. It is advantageous of the present invention over the prior arts that a large variety of polypeptides, preferably antigens, with different length, hydrophobicity and structure can be fused at either terminus of the coat protein of AP205 and the resulting fusion proteins still retain the capability of forming virus-like particles. For example, we have found that a fusion protein comprising the coat protein of AP205 and a highly hydrophobic T-cell epitope, the p33 epitope, forms virus-like particles; in contrast, a fusion protein comprising the same T-cell epitope and the coat protein of fr fails to form VLPs. Furthermore, the antigens that are fused to the coat protein acquire the proper folding and are displayed on the outer surface of the virus-like particles, and the modified VLPs induce strong antibody responses against the antigens.

The present invention further advantageously allows the free accessibility of at least one end of the at least one antigen, which is of importance if the free end accounts for the induction of a strong immune response. Moreover the possibility to use the same VLP to display the at least one antigen at either end of the coat protein allows evaluation of the immunogenicity of the N- or the C-terminal fused to at least one antigen, independent of carrier effects.

In one preferred embodiment, the modified VLP further comprises at least one immunostimulatory substance, an immunostimulatory nucleic acid, even more preferably an immunostimulatory nucleic acid comprising at least one unmethylated CpG motif. The inclusion of immunostimulatory substance bound to, preferably packaged inside, the modified VLP enhances the immune response induced by the modified VLP. This is of particular advantage if the at least one antigen is a antigen derived from a micropathogen, such as a viral antigen, a bacterial antigen, or an antigen that induces an immune response against cancer cells or against an allergen.

In another aspect, the invention provides a vaccine composition comprising the modified VLP. Furthermore, the invention provides a method of administering the vaccine to an animal or to a human.

In one aspect, the invention provides a method for producing the modified VLP of the invention, comprising the steps of: (a) (optional) in-frame ligating a nucleotide sequence encoding a spacer with either the first nucleotide sequence encoding the first polypeptide or the second nucleotide sequence encoding the second polypeptide; (b) in-frame ligating said second nucleotide sequence with said first nucleotide sequence, resulting in a third nucleotide sequence encoding said fusion protein; (c) (optional) introducing a stop codon which allows suppression at the 3′ of the first nucleotide sequence; (d) expressing said third nucleotide sequence in a host, preferably under conditions such that the resulting expressed proteins are capable of forming said modified VLPs; and (e) purifying said modified VLPs obtained from step (d).

In another aspect, the invention provides a fusion protein comprising a polypeptide, wherein said polypeptide is fused to either the N or the C-terminus or to both terminus of the coat protein, or a mutein thereof, of AP205. In a further aspect, the invention provides a nucleotide sequence encoding said fusion protein.

In one further aspect, the invention provides a pharmaceutical composition comprising the modified VLP and a pharmaceutical acceptable carrier.

In yet another aspect, the invention provides a method of treating or preventing a disease, a disorder or physiologic conditions in an individual, wherein said method comprises administering to an animal or a human a modified VLP of the invention, or the pharmaceutical composition of the invention or the vaccine composition of the invention.

### BRIEF DESCRIPTION OF THE DRAWINGS

FIG. 1 shows the electronmicrographs of the modified VLPs comprising fusion proteins of the coat protein of AP205 and D2 peptide. D2 was fused to the C-terminus of the coat protein via a spacer GSG (construct 418) or via a spacer GTAGGGSG (construct 420). D2 was fused to the N-terminus of the coat protein via a spacer GSGG (construct 421) or via a spacer GSGTAGGGSGS (construct 422).

FIG. 2 shows the inhibition ELISA of the modified VLPs comprising fusion proteins of the coat protein of AP205 and D2 peptide. ♦ construct 418; □ construct 420; □ construct 421; ∘ construct 422. These constructs have been described in FIG. 1.

FIG. 3 shows the electronmicrograph of the modified VLP comprising fusion protein of the coat protein of AP205 and Nef 55. Nef 55 was fused to the C-terminus of the coat protein via a spacer GTAGGGSG.

### DETAILED DESCRIPTION OF THE INVENTION

### Definitions

The term “AP205 bacteriophage” and the term “RNA phage AP205” are interchangeably used herein.

Antigen: As used herein, the term “antigen” refers to a molecule capable of being specifically bound by an antibody or by a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant or require that the antigen is presented in accordance with the present invention. An antigen can have one or more epitopes or antigenic sites (B- and T-epitopes). The term “specifically bound,” as used herein, is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. However, the term “antigen”, as used within the context of this application, refers to an antigen not being the coat protein, or a mutein thereof, of AP205 and not being the VLP of AP205 bacteriophage, rather in addition to the coat protein, or a mutein thereof, of AP205 and in addition to the VLP of AP205 bacteriophage.

Antigenic site: The term “antigenic site” and the term “antigenic epitope,” which are used herein interchangeably, refer to continuous or discontinuous portions of a polypeptide, which can be bound immunospecifically by an antibody or by a T-cell receptor within the context of an MHC molecule. At least in some instances the binding of the antigenic site with antibody requires that the antigenic site is presented in accordance with the present invention. Immunospecific binding excludes non-specific binding but does not necessarily exclude cross-reactivity. An antigenic site typically comprises 5-10 amino acids in a spatial conformation which is unique to the antigenic site.

Bound: As used herein, the term “bound” refers to binding that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term also includes the enclosement, or partial enclosement, of a substance. The term “bound” is broader than and includes terms such as “coupled,” “fused,” “enclosed,” “packaged” and “attached.”

Packaged: The term “packaged” as used herein refers to the state of an immunostimulatory substance, preferably of an immunostimulatory nucleic acid, in relation to the modified VLP. The term “packaged” as used herein, refers to the enclosement, or partial enclosement, of the immunostimulatory substance, preferably of the immunostimulatory nucleic acid substance. The term “packaged” as used herein includes binding that may be covalent, e.g., by chemically coupling, or non-covalent, e.g., ionic interactions, hydrophobic interactions, hydrogen bonds, etc. However, the immunostimulatory substance such as the unmethylated CpG-containing oligonucleotide can be enclosed by the VLP even without the existence of an actual covalent binding. In preferred embodiments, the immunostimulatory nucleic acid is packaged inside the VLP and thus typically and preferably not accessible to DNase or RNase hydrolysis.

Coat protein of AP205 bacteriophage: The term “coat protein of AP205”, as used herein, refers to the coat protein encoded by the genome of AP205 bacteriophage or by the genome of a variant of AP205 bacteriophage. Typically and preferably, the term “coat protein of AP205”, as used herein, refers to the coat protein encoded by the genome of AP205 bacteriophage. More preferably the term “coat protein of AP205” refers to SEQ ID NO: 1 or the amino acid sequence, wherein the first methionine is cleaved from SEQ ID NO:1 (SEQ ID NO:67). Typically and preferably a coat protein of AP205 is capable of assembling as one subunit of a virus capsid or a VLP of RNA phage AP205.

CpG: As used herein, the term “CpG” refers to an oligonucleotide which contains an unmethylated cytosine, guanine dinucleotide sequence (e.g. “CpG DNA” or DNA containing a cytosine followed by guanosine and linked by a phosphate bond) and stimulates/activates, e.g. has a mitogenic effect on, or induces or increases cytokine expression by, a vertebrate cell. For example, CpGs can be useful in activating B cells, NK cells and antigen-presenting cells, such as monocytes, dendritic cells and macrophages, and T cells. The CpGs can include nucleotide analogs such as analogs containing phosphorothioester bonds and can be double-stranded or single-stranded. Generally, double-stranded molecules are more stable in vivo, while single-stranded molecules have increased immune activity.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

A fragment of a coat protein of AP205: The term “a fragment of a coat protein of AP205,” as used herein, refers to a polypeptide that is capable of forming a virus-like particle of AP205 and has at least one truncation, at least one internal deletion or a combination thereof, of a coat protein of AP205 and, moreover has at least 70%, preferably 80% the length of a coat protein of AP205. The term “a fragment of a coat protein of AP205,” as used herein, further encompasses a polypeptide that is capable of forming a virus-like particle of AP205 and has more than 80%, more preferably more than 90% and even more preferably more than 95% amino acid sequence identity to “a fragment of a coat protein of AP205” as defined above.

Fusion (or its verb fuse): As used herein, the term “fusion” (or the verb form, “fuse”) refers to the combination of amino acid sequence of different origin in one polypeptide chain by in-frame combination of their coding nucleotide sequences. More than one nucleotide sequence may encode one given amino acid sequence due to the degeneracy of the genetic code.

Immunostimulatory nucleic acid: As used herein, the term immunostimulatory nucleic acid refers to a nucleic acid capable of inducing and/or enhancing an immune response. Preferably, immunostimulatory nucleic acid contains at least one CpG motif e.g. a CG dinucleotide in which the C is unmethylated. The CG dinucleotide can be part of a palindromic sequence or can be encompassed within a non-palindromic sequence. Immunostimulatory nucleic acids not containing CpG motifs as described above encompass, by way of example, nucleic acids lacking CpG dinucleotides, as well as nucleic acids containing CG motifs with a methylated CG dinucleotide. The term “immunostimulatory nucleic acid” as used herein should also refer to nucleic acids that contain modified bases such as 4-bromo-cytosine.

Immunostimulatory substance: As used herein, the term “immunostimulatory substance” refers to a substance capable of inducing and/or enhancing an immune response. Preferably immunostimulatory substance refers to toll-like receptor activating substances. The term “immunostimulatory substance”, as used within the context of this application, refers to an immunostimulatory substance not being the modified VLP of the present invention, rather in addition to said modified VLP.

A mutein of a coat protein of AP205 bacteriophage: The term “a mutein of a coat protein of AP205 bacteriophage,” as used herein, refers to a polypeptide, the amino acid sequence of which differs by at least one amino acid with respect to an amino acid sequence of a given coat protein of AP205 bacteriophage and the amino acid sequence of which has at least 90%, more preferably 92%, even more preferably 95%, still more preferably 97% identity to the amino acid sequence of a given coat protein of AP205 bacteriophage. Typically and preferably a mutein of a coat protein of AP205 bacteriophage retains the capability of forming a VLP of RNA phage AP205. The term “a mutein of SEQ ID NO:1 or SEQ ID NO:67,” as used herein, refers to a polypeptide, the amino acid of which differs by at least one amino acid with respect to an amino acid sequence of SEQ ID NO:1 or SEQ ID NO:67 and the amino acid sequence of which has at least 90%, more preferably 92%, even more preferably 95%, still more preferably 97% identity to the amino acid sequence of a SEQ ID NO:1 or SEQ ID NO:67. Typically and preferably the sequence difference between the coat protein of AP205 and the mutein of said coat protein is introduced by at least one genetic engineering, wherein said genetic engineering is selected from the group consisting of internal addition, insertion, deletion, truncation (refers to deletion from the end of the protein), substitution and a combination thereof. The externally added amino acid(s), i.e. the added amino acid or amino acids at either or both end of the coat protein of AP205 bacteriophage or the mutein of a coat protein of AP205 bacteriophage is/are not regarded as part of the sequence of the mutein of the coat protein of AP205 bacteriophage. Further preferably at least 50%, preferably at least 70%, more preferably at least 90%, substitutions of the amino acids are conservative amino acid substitutions. Conservative amino acid substitutions, as understood by a skilled person in the art, include, and typically and preferably consist of isosteric substitutions, substitutions where the charged, polar, aromatic, aliphatic or hydrophobic nature of the amino acid is maintained. Typical conservative substitutions are substitutions between amino acids within one of the following groups: Gly, Ala; Val, Ile, Leu; Asp, Glu; Asn, Gln; Ser, Thr, Cys; Lys, Arg; and Phe and Tyr.

Ordered and repetitive antigen array: As used herein, the term “ordered and repetitive antigen array” generally refers to a repeating pattern of antigen, characterized by a typically and preferably high order of uniformity in spatial arrangement of the antigens with respect to the virus-like particle. In one embodiment of the invention, the repeating pattern may be a geometric pattern. The modified VLP of RNA phage AP205 possesses strictly repetitive paracrystalline orders of antigens, preferably with spacings of 1-30 nanometers, preferably 2 to 15 nanometers, even more preferably 2 to 10 nanometers, even again more preferably 2 to 8 nanometers, and further more preferably 1.6 to 7 nanometers, most preferably 0.5 to 7 nanometers.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

Self antigen: The term “self antigen,” as used herein, refers to polypeptides encoded by the host's DNA and products derived from polypeptides or RNA encoded by the host's DNA defined as self. Moreover, the term “self antigen,” as used herein, also preferably refers to polypeptides that comprise, or alternatively consists of, a fraction of a self antigen and, preferably, having a length of at least four, preferably at least five, more preferably at least six, at least seven or at least eight amino acids. In addition, the term “self antigen,” as used herein, also preferably refers to polypeptides that have a high homology to self antigen as defined above, preferably having at least a homology of 80%. Moreover, the term “self antigen,” as used herein, also preferably should encompass orthologs of the self antigen as defined above and the orthologs are capable of generating immune responses specific against the self antigen. The term “ortholog” denotes a polypeptide obtained from one species that is the functional counterpart of a polypeptide from a different species. Sequence differences among orthologs are the result of speciation. Furthermore, the term “self antigen,” as used herein, preferably refers to polypeptides that result from a combination of two or several self antigen.

Virus-like particle (VLP), as used herein, refers to a structure resembling a virus particle. A virus-like particle in accordance with the invention is non-replicative and noninfectious since it lacks all or part of the viral genome, typically and preferably lacking all or part of the replicative and infectious components of the viral genome.

Virus-like particle of RNA phage AP205: As used herein, the term “virus-like particle of a RNA phage AP205,” refers to a virus-like particle comprising, or preferably consisting essentially of or consisting of coat proteins, muteins or fragments thereof, of a RNA phage AP205. In addition, virus-like particle of a RNA phage AP205 resembles the structure of a RNA phage AP205 and is non-replicative and non-infectious, and lacks at least the gene or genes encoding for the replication machinery of the RNA phage AP205, and typically also lacks the gene or genes encoding the protein or proteins responsible for viral attachment to or entry into the host. This definition should, however, also encompass virus-like particles of RNA phage AP205, in which the aforementioned gene or genes are still present but inactive, and, therefore, also leading to non-replicative and noninfectious virus-like particles of RNA phage AP205. Preferred VLPs derived from RNA-phages AP205 exhibit icosahedral symmetry and consist of 180 subunits. Within this present disclosure the term “subunit” and “monomer” are interexchangeably and equivalently used within this context.

Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can be readily determined by one of ordinary skill in the art (for example, by Scatchard analysis.)

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

One, a, or an: when the terms “one,” “a” or “an” are used in this disclosure, they mean “at least one” or “one or more” unless otherwise indicated.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

The present invention provides a modified virus-like particle (VLP) comprising at least one fusion protein, wherein said at least one fusion protein comprises, consists essentially of, or consists of: (a) a first polypeptide; and (b) a second polypeptide; and wherein the first polypeptide is a coat protein, or a mutein thereof, of AP205 bacteriophage, and wherein the second polypeptide is fused to the first polypeptide either to the N- or to the C-terminus of the first polypeptide.

RNA bacteriophage AP205 has been recently identified (Klovins, J., et al., J. Gen. Virol. 83: 1523-33 (2002)). The AP205 RNA phage (Taxonomy ID: 154784) is a single-stranded, positive-strand RNA virus. The AP205 genome is 4267 nucleotides (nt) in length (accessions AF334111, NC_{—}002700). The natural host of the AP205 phage is Acinetobacter spp. (Klovins, J., et al., J. Gen. Virol. 83: 1523-33 (2002)). The genome of the AP205 phage comprises three large open reading frames (ORFs), which code for the maturation, the coat and the replicase proteins, respectively. In addition, two additional small ORFs are present at the 5′ terminus, preceding the maturation gene. The function of the proteins coded by these ORFs is unknown. It has been postulated that one of these ORFs might code for a lysis protein (Klovins, J., et al., J. Gen. Virol. 83: 1523-33 (2002)). Assembly of AP205 coat protein expressed in E. coli into a VLP has also been recently disclosed in WO 2004/007538 the disclosure of which is herein incorporated by way of reference. The cloning and expression the coat protein of AP205 have been disclosed from the second paragraph of page 24 to the second paragraph of page 25 of WO 04/007538 and in EXAMPLE 1 and 2 of the same application and these specific disclosures are also herein incorporated by way of reference.

In one preferred embodiment, the first polypeptide consists of 118-144 amino acids, preferably 121-141, more preferably 124-138, even more preferably 127-135, still further preferably 128-134 amino acids. In one further preferred embodiment, the first polypeptide consists of 131-142 amino acids, preferably 131-139, more preferably 131-135 and still more preferably 131-134 amino acids.

In one preferred embodiment of the invention, the coat protein, or a mutein thereof, of AP205 bacteriophage is selected from a group consisting of: (a) SEQ ID NO:1; (b) SEQ ID NO:2; (c) SEQ ID NO:42; (d) SEQ ID NO:67; (e) SEQ ID NO:68; (f) SEQ ID NO:69 and (g) a mutein of SEQ ID NO:1, or 67. In one preferred embodiment, the mutein has the amino acid sequence as set forth in SEQ ID NO:1, 2, 42, 67, 68 or 69, wherein at most six amino acid residues, preferably at most five, four or three amino acid residues, more preferably at most two amino acid residues, and even more preferably one amino acid residue of SEQ ID NO:1, 2, 42, 67, 68 or 69 is, deleted, internally added, or substituted, wherein preferably at least one, more preferably at least two, three or four, and even more preferably all of said substitutions are conservative substitutions.

In one preferred embodiment, the mutein has the amino acid sequence as set forth in SEQ ID NO:1, 2, 42, 67, 68 or 69, wherein at least one cysteine residue, preferably at most two cysteine residues, is deleted or substituted, wherein preferably said at least one, more preferably at least two, and even more preferably all of said substitutions are conservative substitutions.

In one preferred embodiment, the mutein has the amino acid sequence as set forth in SEQ ID NO:1, 2, 42, 67, 68 or 69, wherein at least one lysine residue, preferably at most three lysine residues, more preferably at most two lysine residues, and even more preferably one lysine is deleted or substituted, wherein preferably said at least one, more preferably at least two or three, and even more preferably all of said substitutions are conservative substitutions.

This invention is based on the surprising finding that a large variety of polypeptides with various sequences and various lengths can be fused to the coat protein, or a mutein thereof, of AP205 bacteriophage and the resulting fusion proteins retain the capability of forming VLP.

In one preferred embodiment, the second polypeptide has less than 100, more preferably less than 80, less than 60, more preferably less than 40, still more preferably less than 30 amino acids. Preferably the second polypeptide folds into an independent domain or folding unit which does not interfere with the assembly of the coat protein, or a mutein thereof, of AP205 bacteriophage, into a VLP.

In one further preferred embodiment, the second polypeptide consists of 1-60 amino acids, preferably 3-40, more preferably 5-30, still more preferably 10-25 amino acids. Preferably the presence of the second polypeptide does not interfere with the assembly of the first polypeptide into a VLP.

In one preferred embodiment, the second polypeptide comprises or alternatively consists of, at least one amino acid with at least one reactive functional group. In one further preferred embodiment, the second polypeptide comprises or alternatively consists of, at least one cysteine, preferably one cysteine residue. The at least one amino acid with at least one reactive functional group is useful as attachment site for associating with other functional groups comprised by the same or by other molecular moieties.

In one further aspect, the invention provides the use of the modified VLP as a protein-based drug delivery system, wherein said drug is packaged inside of the modified VLP. Drug refers to, typically and preferably chemical compounds, toxins, biologically active substances, nucleic acids for gene therapy purpose. In one further preferred embodiment, the second polypeptide comprises a target molecule which is a polypeptide. Protein-based drug delivery systems have been disclosed in prior arts, such as Brown W L. et al. (2002) Intervirology 45: 371.380, Wu M. et al. (1995) Bioconjugate Chem. 6: 587-595 and in European Patent No. EP 0 648 272. These disclosures are incorporated herein by reference in their entireties.

In one preferred embodiment of the invention, the second polypeptide comprises, alternatively consists essentially of, or consists of at least one antigen, wherein the at least one antigen is a polypeptide. The size, hydrophobicity the structure of the antigen should be compatible with the assembly of the fusion protein into a VLP in accordance with the present invention. This invention is further based on the surprising finding that a large variety of antigens with various sequences and various lengths can be fused to the coat protein, or a mutein thereof, of AP205 bacteriophage and the resulting fusion proteins retain the capability of forming VLP. Furthermore, it is surprisingly found that the antigen or at least one antigenic site of the antigen is displayed on the outer surface of the formed VLP.

Assembly of the fusion protein into a VLP may be tested, as one skilled in the art would appreciate by expressing the fused coat protein in E. coli, optionally purifying the capsids by gel filtration from cell lysate, and analysing the capsid formation in an immunodiffusion assay (Ouchterlony test) or by Electron Microscopy (EM) (Kozlovska, T. M. et al., Gene 137:133-37 (1993)). Immunodiffusion assays and EM may be directly performed on cell lysate.

The display of the antigen or at least one antigenic site of the antigen on the surface of the modified VLP may be assessed by immunizing an animal, such as a mouse, with the modified VLPs and determining the antibody response in an ELISA specific for the antigen or for at least one antigenic site of the antigen. Alternatively an inhibition ELISA may be performed. The antigen is directly or indirectly coated on an ELISA plate. The inhibition of the binding of an antigen-specific serum, e.g. a mouse serum, to the coated antigen can be determined by adding a serial of dilutions of the modified VLPs.

In one embodiment, the at least one antigen is a protein. In another embodiment, the at least one antigen is a fragment of a protein. The term “a fragment of a protein”, or its interchangeably used term “a fragment of a polypeptide” or “a fragment of an antigen”, as used herein, should encompass any polypeptide comprising, or alternatively or preferably consisting of, at least 6, 7, 8, 9, 10, 11, 12, 17, 18, 19, 20, 25, 30 contiguous or discontinuous amino acids of the protein, polypeptide or antigen, as defined herein, as well as any polypeptide having more than 65%, preferably more than 80%, more preferably more than 90% and even more preferably more than 95% amino acid sequence identity thereto. A fragment of a protein should comprise at least one antigenic site. A fragment of a protein, when presented in accordance with the present invention, should be capable of inducing the production of antibody, or stimulation of T cell, in vivo, which specifically binds to the protein or to a fragment of the protein presented in the context of the MHC molecule. Preferred embodiments of a fragment of a protein are truncation or internal deletion forms of the protein.

Methods to determine antigenic site(s) of a protein are known to the skilled person in the art. PCT/EP2005/004980, has elaborated some of these methods from the first paragraph of page 26 to the fourth paragraph of page 27 therein, and these specific disclosures are incorporated herein by reference. It is to be noted that these methods are generally applicable to other polypeptide antigens, and therefore are not restricted to IL-23 p19 as disclosed in PCT/EP2005/004980.

In still another embodiment of the invention, the at least one antigen is a variant of a protein. The term “a variant of a protein” or its interchangeably used term “a variant of a polypeptide” or “a variant of an antigen,” as used herein, should encompass any polypeptide comprising, or alternatively or preferably consisting of, any natural or genetically engineered polypeptide having more than 70%, preferably more than 80%, even more preferably more than 90%, again more preferably more than 95%, and most preferably more than 97% amino acid sequence identity with the sequence of the protein, antigen or polypeptide. Preferred methods of generating a variant of a protein is by genetic engineering, preferably by insertion, substitution, deletion or a combination thereof. A variant of a protein, when presented in accordance with the present invention, should be capable of inducing the production of antibody, or stimulation of T cell, in vivo, which specifically binds to the protein or to a fragment of the protein presented in the context of the MHC molecule.

In one preferred embodiment, the second polypeptide comprises or alternatively consists of at least one naturally-occurring antigen, or a portion thereof, wherein the portion of the naturally-occurring antigen comprises or alternatively consists of at least one antigenic site. Naturally-occurring antigen refers to an antigen, the amino acid sequence of which exists in nature, preferably exists in an organism, such as a plant, an animal, a microorganism, such a bacteria or a virus.

In preferred embodiments of the invention, the at least one antigen is selected from a group consisting of: (a) an antigen suited to induce an immune response against cancer cells; (b) an antigen suited to induce an immune response against at least one microbial pathogen; (c) an antigen suited to induce an immune response against at least one allergen; (d) an antigen suited to induce an immune response against at least one self antigen; (e) an antigen suited to induce an immune response in farm animals or pets; and (f) an antigen suited to induce a response against a polypeptide toxin or a polypeptide hormone.

In one preferred embodiment of the invention, the at least one antigen is suited to induce an immune response against cancer cells; preferably the at least one antigen is a tumor antigen, a variant or a fragment thereof. In one further preferred embodiment of the invention, the tumor antigen is a polypeptide of breast cancer cells, a polypeptide of kidney cancer cells, a polypeptide of prostate cancer cells, a polypeptide of skin cancer cells, a polypeptide of brain cancer cells, or a polypeptide of leukaemia cells. In one still further preferred embodiment, the tumor antigen is selected from the group consisting of: (a) Her2; (b) GD2; (c) EGF-R; (d) CEA; (e) CD52; (f) human melanoma protein gp100; (g) human melanoma protein melan-A/MART-1; (h) tyrosinase; (i) NA17-A nt protein; (j) MAGE-3 protein; (k) p53 protein; (l) CD21; (m) HPV16 E7 protein; (n) fragments of any of the tumor antigens from (a) to (m); and (o) variants of any of the tumor antigens from (a) to (m).

In another preferred embodiment of the invention, the at least one antigen is suited to induce an immune response against an infectious diseases or against at least one microbial pathogen; preferably the at least one antigen is an antigen derived from a microbial pathogen, or a variant or a fragment thereof. Infectious diseases, microbial pathogens, and antigens that derived from the microbial pathogens have been disclosed in US patent application US-2003-0091593-A1, in particular from the second paragraph of page 75 to the fourth paragraph of page 83. These disclosures are herein incorporated by way of reference. In one further preferred embodiment, the at least one antigen derived from microbial pathogens is a polypeptide of HIV, a polypeptide of Influenza virus, a polypeptide of Hepatitis B virus, Hepatitis C virus, a polypeptide of Toxoplasma, a polypeptide of Plasmodium falciparum, a polypeptide of Plasmodium vivax, a polypeptide of Plasmodium ovale, a polypeptide of Chlamydia, a polypeptide of Plasmodium malariae or an Influenza M2 protein, variants of the afore-mentioned polypeptides, and fragments of the afore-mentioned polypeptides. Therefore in one aspect, the invention provides the use of the modified VLP of the invention as a vaccine to prevent and/or treat infectious diseases. The vaccine can be administered to an animal or a human. Preferred animal is a farm animal or a house pet, for example, but not limited to a pig, a horse, a cow, a sheep, a dog, a cat, a rabbit and a chicken.

In one preferred embodiment, the at least one antigen comprises or consists essentially of, or consists of the extracellular domain of the Influenza M2 protein. In one preferred embodiment, the extracellular domain of the M2 protein is fused to the N-terminus of the coat protein, muteins or fragments there of, of the AP205 bacteriophage. In one preferred embodiment, the at least one antigen comprises, or consists essentially of, or alternatively consists of a fragment of the extracellular domain of the Influenza M2 protein, wherein said fragment has at least 5, preferably at least 7, more preferably at least 10 consecutive amino acids out of the sequence of the extracellular domain of the Influenza M2 protein. In a further preferred embodiment, said fragment of the extracellular domain of the Influenza M2 protein comprises the N-terminus half of said extracellular domain. In one preferred embodiment, the extracellular domain of the Influenza M2 protein consists of the sequence as of SEQ ID NO:43. In one further preferred embodiment, the extracellular domain of the Influenza M2 protein consists of the sequence as of SEQ ID NO:43, wherein at most three amino acid residues of SEQ ID NO:43 are deleted, internally added, or substituted.

In one preferred embodiment, the at least one antigen comprises or consists essentially of, or consists of M2 protein in tandem, preferably M2 dimers in tandem, or alternatively M2 trimers in tandem. This embodiment may enhance the immune responses elicited against the M2 peptide. In one further preferred embodiment, the at least one antigen further comprises at least one spacer, wherein said spacer is positioned between the M2 peptides. Preferably the spacer has at most 15 amino acids, preferably at most 10, more preferably at most 8, at most 6, more preferably at most 4 amino acids.

In one preferred embodiment of the invention, the at least one antigen comprises or consists of PreS1(aa21-47), a peptide derived from the PreS1 region of the Hepatitis B virus (HBV) large surface protein (PLGFFPDHQLDPAFRANTANPDWDFNP, SEQ ID NO:62). The envelope of human HBV contains three coterminal proteins, designated small (S), middle (M), and large (L) surface protein. The S protein is the most abundant of the three and consists of 226 amino acids. The M protein comprises the S protein sequence and an additional 55 amino acids at the N-terminus. The 55 amino acid sequence is designated as the PreS2 sequence. The L protein comprises the S and PreS2 sequences and an additional 119 (or 108, depending on the HBV subtype) amino acids at the N-terminus, which is designated as the PreS1 sequence. It has been shown that the HBV-binding site for the yet to be identified hepatocyte receptor is located within the PreS1 region, between amino acids 21 and 47 (Shouval, D., 2003, Journal of Hepatology 39. S70-S76).

Hepatitis B is a major health problem, with more than 350 million people chronically infected worldwide. Chronic infection with Hepatitis B virus leads to a number of diseases, including liver cirrhosis and cancer.

In one preferred embodiment, the at least one antigen is a HIV protein, fragments or variants thereof. Useful HIV antigens includes p17-GAG, p24-GAG, p15-GAG, Protease, reverse transcriptase (RT), Integrase, Vif, Vpr, Vpu, Tat, Rev, gp-41-Env, gp-120-Env and Nef (Addo, M. M. et al., J. Virol. 77: 2081-2092 (2003)). The HIV antigens p24-GAG and Nef have been found to have the highest epitope density (Addo, M. M. et al., J. Virol. 77: 2081-2092 (2003)). In preferred embodiments of the invention, the antigen comprises therefore p24-GAG-CTL and/or NEF-CTL and/or Th cell epitopes. Th cell epitopes are believed to contribute to the induction and maintenance of CTL responses. HIV CTL epitopes and HIV consensus sequences can be selected from the database (e.g. website: http:/hiv-web.lanl.gov/seq-db.html) and from the reference “The Identification of Optimal HIV-Derived CTL Epitopes in Diverse Populations Using HIV Clade-Specific Consensus” (, pp. I-1-20 in HIV Molecular Immunology 2001. Edited by: Korber B T K, Brander C, Haynes B F, Koup R, Kuiken C, Moore J P, Walker B D, and Watkins D.) The T-cell response induced upon vaccination is assessed in proliferation assays (for Th cell response, Belshe R. B. et al., J. Inf. Dis. 183: 1343-1352 (2001)), in ELISPOT assays (Oxenius, A. et al., Proc. Natl. Acad. Sci. USA 99: 13747-13752 (2002)), or in Cytotoxicity assays (Belshe R. B. et al., J. Inf. Dis. 183: 1343-1352 (2001)).

In one preferred embodiment, the at least one antigen comprises or consists of a polyepitope of HIV. The term “polyepitope of HIV” as used herein shall refer to a combination of at least two HIV epitopes, derived from the same or different HIV polypeptides, wherein said at least two HIV epitopes are fused into one polypeptide. In a further preferred embodiment, the polyepitope is fused to the C-terminus of AP205 coat protein, or a mutein thereof. In one preferred embodiment, the at least one antigen is a polyepitope derived from HIV Nef. In again a preferred embodiment, the polyepitope derived from Nef is Nef 55 (SEQ ID NO:23). In a still further preferred embodiment, the at least one antigen Nef55 is fused to the C-terminus of AP205 coat protein. In another preferred embodiment, the at least one antigen is a polyepitope derived from HIV Gag. In one further preferred embodiment, the polyepitope derived from HIV Gag is gag G50 (SEQ ID NO:119). In one further preferred embodiment, the polyepitope derived from HIV Gag is gag G50 with addition lysine residue at the C terminus to increase the solubility of the peptide (SEQ ID NO: 120).

In one preferred embodiment, the at least one antigen comprises, consists essentially of, or consists of a peptide derived from the HIV envelope glycoprotein gp160, wherein preferably said peptide is highly conserved among all HIV strains (more than 70% conservative) or wherein preferably said peptide induces neutralizing antibodies or wherein preferably said peptide is a blocking peptide. In one further preferred embodiment, the peptide derived from the HIV envelope glycoprotein gp120 or gp41 is selected from the group consisting of:

| | | | |
|---|---|---|---|
| (a) | HIV env 1: | SLEQIWNNMTWMQWDK; | (SEQ ID NO:98) |
| (b) | HIV env 2: | SLEQIWNNMTWMQWDR; | (SEQ ID NO:99) |
| (c) | HIV env 3: | IWNNMTWMQWDR; | (SEQ ID NO:100) |
| (d) | HIV env 4: | WASLWNW; | (SEQ ID NO:101) |
| (e) | HIV env 5: | NWFDISNWLW; | (SEQ ID NO:102) |
| (f) | HIV env 6: | LLELDKWASLWNWFNL; | (SEQ ID NO:103) |
| (g) | HIV env 7: | ELDKWA,; | (SEQ ID NO:104) |
| (h) | HIV env 8: | WMEWDREINNYTSLIHSLI | (SEQ ID NO:105) |
| | | EESQNQQEKNEQELL; | |
| (i) | HIV env 9: | CSKLIC; | (SEQ ID NO:106) |
| (j) | HIV env 10: | GFLGAAGSTMGAASITL | (SEQ ID NO:107) |
| | | VQ; | |
| (k) | HIV env 11: | QQNNLLRAIEAQQHLLQLT | (SEQ ID NO:108) |
| | | VWGIKQL; | |
| (l) | HIV env 12: | GIVQQQ; | (SEQ ID NO:109) |
| (m) | HIV env 13: | QLLGIWGCSGKLICTTAVP | (SEQ ID NO:110) |
| | | WNSSWS; | |
| (N) | HIV env 14: | NAKTIIVQLNQSVE; | (SEQ ID NO:111) |
| (O) | HIV env 15: | GGNSNNESEIFRPGGGD; | (SEQ ID NO:112) |
| AND | | | |
| (p) | HIV env 16: | VAPTKAKRRVVQREKRAVG | (SEQ ID NO:113) |
| | | IGALFLGFLGAAGSGC. | |

In one preferred embodiment of the invention, the at least one antigen is suited to induce an immune response against at least one self antigen, preferably the at least one antigen is a self antigen, a variant or a fragment thereof. Examples of diseases, particularly, autoimmune diseases, chronic inflammatory diseases, caused by the overproduction or malfunction of a self antigen have been disclosed in the last paragraph in page 53 of the patent application WO 02/056905.

In one further preferred embodiment of the invention, the self antigen is selected from a group consisting of: (a) lymphotoxins (preferably Lymphotoxin α (LT α), Lymphotoxin β (LT β)); (b) lymphotoxin receptors; (c) receptor activator of nuclear factor kB ligand (RANKL); (d) vascular endothelial growth factor (VEGF); (e) vascular endothelial growth factor receptor (VEGF-R); (f) Interleukin-5; (g) Interleukin-17; (h) Interleukin-13; (i) IL-23 p19; (j) Ghrelin; (k) CCL21; (l) CXCL12; (m) SDF-1; (n) M-CSF; (o) MCP-1; (p) Endoglin; (q) GnRH; (r) TRH; (s) Eotaxin; (t) Bradykinin; (u) BLC; (v) Tumor Necrosis Factor α; (w) amyloid beta peptide (Aβ₁₋₄₂); (x) Aβ₁₋₆; (y) Angiotensin; (z) CCR5 extracellular domain; (aa) CXCR4 extracellular domain; (bb) Gastrin; (cc) CETP; (dd) C5a; (ee) Bradykinin; (ff)) Des-Arg Bradykinin; (gg) fragments of (a) off); (hh) variants of (a)-(ff). Detailed description of the afore-mentioned self antigens, fragments or variants thereof, have been disclosed in WO 02/056905 from the last paragraph of page 56 to the first paragraph of page 86. These disclosures are incorporated herein by way of reference.

In one preferred embodiment, the at least one antigen is an IL-23 p19 protein, or more preferably an IL-23 p19 fragment, as described in PCT/EP2005/004980, which is incorporated herein by reference in its entirety. Particular preferred fragments useful for the present invention are SEQ ID NO:4-15, SEQ ID NO:52 and 53 disclosed in PCT/EP2005/004980.

In one preferred embodiment, the at least one antigen is a GnRH, or a fragment thereof. VLP-GnRH conjugates useful in the production of vaccines are disclosed in PCT/EP2005/053858, which is incorporated herein by reference in its entirety. In a preferred embodiment, GnRH (EHWSYGLRPG (SEQ ID NO:20) or QHWSYGLRPG (SEQ ID NO:114)), is fused at the C-terminus of the coat protein of AP205. This modified VLP comprising GnRH as the at least one antigen can be administered to a mammal, such as pig to prevent the boar taint in the meat. This modified VLP comprising GnRH can be administered to an animal, such as dog, cat, sheep, cattle to control their reproductive behaviour and/or to reduce their reproductivity. This modified VLP comprising GnRH can be administered to human having gonadal steroid hormone dependent cancers.

In one preferred embodiment, the at least one antigen is ghrelin or a variant or a fragment thereof. VLP-ghrelin conjugates useful in the production of vaccines for the treatment of obesity and other disease associated with increased food-uptake or increased body weight have been disclosed in PCT patent application publication no. WO 04/009124, the disclosure of which is incorporated herein by reference in its entirety. Particularly preferred ghrelin peptides or fragments useful for the present invention are SEQ ID NO: 31-32, 48-56, 59-63 and 111-119 of WO 04/009124, as well as a cat ghrelin, a variant or a fragment thereof, a dog Grehlin, and a variant or a fragment thereof. In one further preferred embodiment, the at least one antigen is a ghrelin having amino acid sequence of human ghrelin as of SEQ ID NO:54 or its corresponding orthologs from other mammals, such as dog or cat. In one further preferred embodiment, the at least one antigen is a ghrelin fragment comprising amino acid 3-7 of SEQ ID NO:54. Preferably said ghrelin fragment has at most 28, preferably at most 25, more preferably at most 20 amino acids in total. In one further preferred embodiment, the ghrelin fragment comprising amino acid 3-7 of SEQ ID NO:54 and comprising or preferably consisting of 18 contiguous amino acids, preferably 16 contiguous, more preferably 14 contiguous amino acids of SEQ ID NO:54. In one preferred embodiment, the ghrelin fragment comprising or alternatively consisting of amino acid as of SEQ ID NO:55. In one preferred embodiment, the ghrelin fragment comprising or alternatively consisting of amino acid sequence as of SEQ ID NO:56. In one preferred embodiment, the ghrelin fragment comprising or alternatively consisting of amino acid sequence as of SEQ ID NO:57. In one preferred embodiment, the ghrelin fragment comprising or alternatively consisting of amino acid sequence as of SEQ ID NO:58 for dog and SEQ ID NO:59 for cat.

In one specific preferred embodiment, the at least one antigen comprises or consists of an angiotensin, a variant or a fragment thereof. VLP-angiotensin conjugates useful in the production of vaccines for the treatment of high blood pressure have been disclosed in PCT patent application publication no. WO 03/031466, which is incorporated herein by reference in its entirety. Particularly preferred protein or fragments useful for the present invention are Angio I: DRVYIHPF (SEQ ID NO:15), Angio XVIII: DRVYIHP (SEQ ID NO:115) and Angiotensin I: DRVYIHPFHL (SEQ ID NO:116). In one preferred embodiment of the invention, Angio I is fused to the C-terminus of AP205 coat protein.

In one specific preferred embodiment, the at least one antigen comprises or consists of an amyloid beta peptide fragments. One particularly preferred such fragment is Aβ1-6 (DAEFRH, SEQ ID NO:117), which is disclosed in PCT patent application publication no. WO 04/016282 which is incorporated herein by reference in its entirety.

In one specific preferred embodiment, the at least one antigen comprises or consists of a TNF-α, a variant or a fragment thereof. Preferred fragments of TNF-α useful for the present invention have been disclosed in PCT/EP2005/005935 and PCT/EP2005/005936. The whole contents of these two applications are incorporated herein by way of reference. In one very preferred embodiment, the at least one antigen is the amino acid 4-23 of mouse TNF-α sequence (SEQ ID NO:41). In one further preferred embodiment, the antigen is fused to the C-terminus of the coat protein of AP205. In one preferred embodiment of the invention, the modified VLP comprising at least one fusion protein, wherein said fusion protein comprises SEQ ID NO:41, is used as a vaccine in a human.

In one preferred embodiment, the said at least one antigen is CXCR4, preferably a CXCR4 extracellular domain, more preferably a fragment of a CXCR4 extracellular domain. The chemokine receptor CXCR4, also known as LESTR or fusin, belongs to the family of seven-transmembrane domain G-protein coupled receptors (Federsppiel et. al. (1993), Genomics 16:707). The only known ligand for CXCR4 is SDF-1 (Pelchen-Mattews, et. al. (1999) Immunol. Rev. 168:33). CXCR4 was later identified as a co-receptor for HIV (Feng et al (1996) Science 272:872). Accordingly, HIV strains that necessity CXCR4 for entry are categorized as X4 strain. SDF-1 has been shown to block HIV-1 entry (Oberlin et al (1996), Nature 382:833; Bleul, et al (1996) Nature 382:829.

In one preferred embodiment of the invention, the at least one antigen comprises or consists of a fragment of a CXCR4 extracellular domain. A fragment of a CXCR4 extracellular domain has at least 6, 7, preferably at least 8, 9, 10 amino acids and a fragment of CCR5 extracellular domain has less than 30, preferably 20, more preferably 15, even more preferably 12 amino acids.

In one preferred embodiment, the at least one antigen comprises or consists of the N-terminal extracellular domain of CXCR4. In one further preferred embodiment, the N-terminal extracellular domain of CXCR4 comprises or consists of SEQ ID NO:48. In one preferred embodiment, the at least one antigen comprises or consists of a fragment of CXCR4 extracellular domain ECL2. Preferably said fragment has at least 6, preferably 7 amino acids. In a further preferred embodiment, the at least one antigen comprises or consists of a fragment of CXCR4 extracellular domain ECL2 having amino acid sequence as SEQ ID NO:49.

In one preferred embodiment of the invention, the at least one antigen is CCR5, preferably a CCR5 extracellular domain, more preferably a fragment of a CCR5 extracellular domain. HIV R5 strains use the cell surface molecules CD4 and CCR5 for attachment and entry into macrophages and CD4+ T cells. CCR5 is a 7-transmembrane receptor with four extracellular domains: an N-terminal sequence and three loops exposed to the extracellular space, which are called subsequently PNt, ECL-1, ECL-2, and ECL-3, respectively.

In one preferred embodiment of the invention, the at least one antigen comprises or consists of a fragment of a CCR5 extracellular domain. A fragment of a CCR5 extracellular domain has at least 6 or 7, preferably at least 8, 9 or 10 amino acids and a fragment of CCR5 extracellular domain has less than 35, preferably less than 30, preferably less than 20, more preferably less than 15, even more preferably less than 12 amino acids.

In one preferred embodiment, the fragment of a CCR5 extracellular domain comprises or consists of ECL2A. ECL2A, as generally understood in the art, starts preferably from the first amino acid of the ECL2 and stops preferably at threonine, which is right before cysteine in ECL2. In one further preferred embodiment, ECL2A comprises or alternatively consists of SEQ ID NO: 46. In one preferred embodiment, the antigen of the invention comprises or consists of CCR5 extracellular domain PNt. In one further preferred embodiment, the PNt domain comprises or preferably consists of SEQ ID NO:45. In one preferred embodiment, the antigen of the invention comprises or consists of CCR5 extracellular domain ECL2. In one further preferred embodiment, the ECL2 domain comprises or preferably consists of SEQ ID NO:91. Preferably or alternatively the naturally-occurring cysteine in the PNt or ECL2 sequence has been substituted by Serine.

In one preferred embodiment of the invention, the at least one antigen is gastrin and/or progastrin. Gastrin (G17) is a group of classical gut peptide hormonese with much lower amount in the colon and pancreas (Koh, Regulatory Peptides. 93, 37-44 (2000)). Gastrin is processed from its precursor progastrin (G34). Both gastrin and progestin exist in a C-terminal glycine-extended form and in a C-terminal phenylalanine amidated form. Gastin is well known for its ability to stimulate gastric acid secretion (Pharmacol Ther. 98, 109-127 (2003)). Recent data suggest that gastrin might promote the development of cancers of the gastrointestinal tract.

In one preferred embodiment, the at least one antigen comprises or preferably consists of G17 (SEQ ID NO:47). In one further preferred embodiment, the at least one antigen comprises or consists of G17 with addition glycine at the C-terminus. In one preferred embodiment, the at least one antigen comprises or consists of progastrin G34 (SEQ ID NO:60). In one further preferred embodiment, the at least one antigen comprises or consists of progastrin G34 with additional glycine at the C-terminus. In one preferred embodiment, the at least one antigen comprises or consists of G17 1-9 fragment (SEQ ID NO:61), preferably with a linker sequence fused to its C-terminus, more preferably with a linker sequence SSPPPPC fused to the C-terminus.

It is to note E at position one of sequence EGPWLEEEE as part of gastrin sequence could be E, pyro E or Q. When additional amino acid is fused to the N-terminus of EGPWLEEEE, E at position one of sequence EGPWLEEEE could be E or preferably Q.

In one preferred embodiment of the invention, the at least one antigen is C5a. C5a, a 74-amino acid, 4-helix bundle glycoprotein (Fernandez and Hugli, J. Biol. Chem. 253, 6955-6964, 1978), is responsible for generating a number of diverse effects on cellular systems, especially neutrophils, endothelial cells and macrophages to induce local inflammations to combat infecting microorganisms (Ward P., Nat. Rev. Immunol. 4:133, 2004). However, by the same token, the excessive generation of C5a in sepsis leads to serious functional defects in neutrophils (Czermak et al., Nat. Med. 5:788, 1999; Huber-Lang et al., J. Immunol. 166:1193, 2001). Elevated activation of C5a has been also implicated in a number of primary and/or chronic inflammatory diseases, such as rheumatoid arthritis (Jose P. Ann Rheum. Dis. 49:747, 1990), psoriasis (Takematsu H., Arch. Dermatol. 129:74, 1993), adult respiratory distress syndrome (Langlois P., Heart Lung 18:71, 1989), reperfusion injury (Homeister, J. Annu. Rev. Pharmacol. Toxicol. 34:17, 1994), lupus nephritis and bullous pemphigoid.

In one preferred embodiment, the at least one antigen comprises or consists of a C5a. In one preferred embodiment, the at least one antigen comprises or consists of a C5a fragment. In one further preferred embodiment, the C5a fragment having amino acid sequence as SEQ ID NO:50.

In one preferred embodiment of the invention, the at least one antigen is CETP. Cholesteryl-ester transfer protein (CETP) is a plasma glycoprotein which mediates the exchange of cholesterol ester (CE) and triglycerides (TG) between High density lipoprotein (HDL) particles and apo B rich particles such as very-low density liporprotein (VLDL) particles or low-density lipoprotein (LDL) particles. Inhibition of CETP activity in rabbits using small molecule inhibitors, anti-sense oligonucleotides or active immunization has consistently shown an anti-atherogenic effect (Barter, P. J. et al. (2003) Arterioscler. Thromb. Vasc. Biol. 23: 160-167).

In one preferred embodiment, the at least one antigen comprises or consists of a CETP fragment having amino acid sequence of SEQ ID NO:51. In one preferred embodiment of the invention, the at least one antigen comprises or consists of Bradykinin. Bradykinin (BK, KRPPGFSPFR, SEQ ID NO:52) is a major vasodilator peptide and plays an important role in the local regulation of blood pressure, blood flow and vascular permeability (Margolius H. S, et al., Hypertension, 1995). Moreover several other biologic activities of Bradykinin have been described including contraction and relaxation of smooth muscles, induction of nociception and hyperalgesia and mediation of inflammatory responses. Bradykinin exerts its effects via the B2-receptor.

In one preferred embodiment of the invention, the at least one antigen comprises or consists of des-Arg9-Bradykinin. des-Arg9-BK (KRPPGFSPF, SEQ ID NO:53) has both overlapping and distinct functions from Bradykinin. Evidence suggests that des-Arg9-BK is rapidly generated after tissue injury and modulates most of the events observed during inflammatory processes including vasodilatation, increase of vascular permeability, plasma extravasation, cell migration, pain and hyperalgesia (Calixto J. B. et al., Pain 2000). Des-Arg9-BK exerts its effects via the B1-receptor. The importance of des-Arg9-BK in inflammatory processes is further emphasized by the observation that B1R−/− mice showed a strongly reduced inflammatory response in a model of acute pleural inflammation (Pesquero J. B. et al., PNAS, 2000).

BK and Des-Arg9-BK play roles in primary and chronic inflammatory diseases, in particular, arthritis and airway inflammation induced by allergens or particulate antigens, such as virus.

In one preferred embodiment of the invention, the at least one antigen is suited to induce an immune response against allergy, preferably the at least one antigen is an allergen, a variant or a fragment thereof. In one further preferred embodiment, the at least one antigen is selected from a group consisting of: (a) a polypeptide involved in bee sting allergy, (b) a polypeptide involved in nut allergy, (c) a polypeptide involved in food allergies, (d) a polypeptide involved in asthma (e) a polypeptide involved in house dust mite allergy; (f) a polypeptide involved in pollen allergies; (e) a variant of (a) to (d); and (f) a fragment of (a) to (d). In one still further preferred embodiment, the at least one antigen is selected from a group consisting of: (a) a phospholipase A₂ protein; (b) Bet v I (birch pollen antigen); (c) Dol mV (white-faced hornet venom allergen); (d) Mellitin; (e) a Der p I peptide (house dust mite allergen), (f) a variant of (a) to (e); and (g) a fragment of (a) to (e).

In one preferred embodiment, the at least one antigen is suited to induce an immune response against a polypeptide toxin, preferably the at least one antigen is a polypeptide toxin, a fragment or a variant thereof.

In one preferred embodiment of the invention, the at least one antigen is suited to induce an immune response against a polypeptide hormone, preferably the at least one antigen is a polypeptide hormone, a fragment or a variant thereof.

In one preferred embodiment of the invention, the at least one antigen is an antigen suited to induce an immune response in a farm animal or a pet. In a further preferred embodiment, the antigen is selected from the group consisting of: (a) an antigen suited to induce an immune response against cancer cells of a farm animal or a pet; (b) an antigen suited to induce an immune response against at least one microbial pathogen infecting a farm animal or a pet; (c) an antigen suited to induce an immune response against at least one self antigen of a farm animal or a pet; and (d) an antigen suited to induce a immune response against a polypeptide toxin or a polypeptide hormone. Examples of antigens that are useful for the present invention as disclosed in this application can be of a human or of an animal origin, the latter are therefore preferred embodiments of the invention when the modified VLP of the invention is used as a vaccine in an animal. The term “animal” is meant to include, for example, humans, sheep, elks, deer, mule minks, monkeys, horses, bulls, cattle, pigs, goats, dogs, cats, chickens, ducks, rats, and mice. Preferred animals are vertebrates, more preferred animals are eutherians, and even more preferred animals are mammals.

In one preferred embodiment of the invention, the fusion protein of the invention further comprises a spacer and wherein said spacer is positioned between said first polypeptide and said second polypeptide. The selection of a spacer will be dependent on the nature of the antigen of the invention, on its biochemical properties, such as pI, charge distribution and glycosylation. In general, flexible spacers are favoured. A spacer is preferably not longer than 30, more preferably not longer than 15 amino acids. Glycine and serine residues are particularly favored amino acids to be used in the spacer, and preferably a spacer comprises at least one glycine or at least one serine residue. Other amino acids, preferably alanine, threonine, and charged amino acids, may be comprised by the spacer. In some cases, proline may also be comprised by the spacer. Spacer is usually added to increase the distance between the coat protein of AP205 and the at least one antigen. Furthermore, a spacer may confer additional flexibility, which may diminish the potential destabilizing effect of fusing the at least one antigen sequence into the sequence of a coat protein of AP205 and diminish the interference with the assembly by the presence of the at least one antigen.

The engineering of the spacer between the first polypeptide and the second polypeptide can be achieved by recombinant DNA technology. For example one convenient method is to incorporate the nucleotide sequence encoding the spacer into the primer sequence used to clone the at least antigen of the invention. Alternatively the nucleotide sequence encoding the spacer may be incorporated into the primer sequence used to clone the coat protein of AP205 into an expression vector, which results in a plasmid expressing the coat protein of AP205 with a spacer fused at the either the N- or the C-terminus.

In one preferred embodiment, the spacer has at most 15 amino acids, preferably at most 13, even more preferably at most 11, still more preferably at most 8 amino acids, further more preferably at most 4, still further more preferably at most 3 amino acids.

In one specific embodiments of the invention, the amino acid sequence of the spacer is selected from a group consisting of: (a) GSGG; (b) GSG; (c) GTAGGGSG; (d) SGG and (e) GSGTAGGGSGS.

In one preferred embodiment, the at least one antigen is flanked by at least one, preferably one cysteine, at each end of the antigen. The flanking cysteines can be naturally occurring within the antigen or artificially added to the antigen. This allows the presentation of the antigen in a circular form by the disulfide bond formed between the two flanking cysteines, which may mimic the naturally existing configuration of the antigen. To avoid undesired disulfide bond formation, the naturally-occurring cysteine(s) within the antigen is preferably to be substituted, preferably by conservative substitution, more preferably by serine, provided that such a substitution does not alter the immunogenecity of the antigen. For example, the ECL2 domain of CCR5 exists in nature as the second extracellular domain of the 7-transmembrane receptor. Thus in one preferred embodiment, the second polypeptide comprises or consists of a cyclic ECL2 or cyclic ECL2a. In one further preferred embodiment, the second polypeptide comprises or consists of SEQ ID NO: 73, SEQ ID NO: 116 or SEQ ID NO:74.

The formation of disulfide bond between the two cysteines flanking the antigen can be promoted by purifying the modified VLP in the presence of 10 mM DTT and subsequently dialyzing against an oxidation buffer which has a pH allowing disulfide bond formation (pH 6.5 to 9, preferably 6.8 to 8.5, for example 50 mM Tris, 150 mM Nacl, pH 8.0) and contains a mixture of oxidized and reduced glutathion (redox shuffle), or other agents catalyzing disulfide bond formation such as a redox shuffle of cystine and cysteine. The redox shuffle may contain for example 0.1 to 5 mM reduced glutathion and 0.1 to 5 mM oxidized glutathion. Preferably, the shuffle is oxidizing. Useful ratios of oxidized to reduced glutathion are for example (in mM) 5/0.2, 5/0.5, 5/1, 5/2, 1/1, 2/2, 1/0.2, 1/0.5, 2/0.2, 2/0.5, 2/1. In an alternative method, the cysteines are reacted with oxidized glutathion (for example 1 to 50 mM) or sodium tetrathionate (e.g. 5 mM), dialyzed to remove excess reagent, and the intra-loop disulfide bond is closed in a disulfide exchange reaction catalyzed by, for example, reduced glutathion (0.1-5 mM), dithiothreitol (0.1-10 mM), beta-mercaptoethanol (0.1-10 mM) or cysteine (0.1-10 mM). Subsequently, the oxidized VLP preparation is dialyzed further against 50 mM Tris, 150 mM NaCl, pH 8.0 or PBS or 20 mM Hepes, 150 mM NaCl, pH 7.2, and may be injected into mice to test immunogenicity of the displayed epitope and the specificity of the elicited antibodies.

The optimal condition for the formation of disulfide bond is tested empirically, by using antibodies specific for the peptide in a loop conformation. In one experimental set-up, the modified VLPs displaying the peptide to be oxidized to a loop conformation is coated on an ELISA plate, then treated on the plate with various buffer conditions as described above, and finally assayed by ELISA with an antibody specific for the peptide in its loop conformation.

In one preferred embodiment, the second polypeptide comprising, consisting essentially of, or consisting of, an amino acid sequence selected from the group consisting of: (a) Influenza virus M2 peptide (SEQ ID NO:43); (b) Hepatitis B virus Pre S1 peptide (SEQ ID NO:62); (c) HIV Nef Polyepitops (SEQ ID NO:23); (d) GnRH (SEQ ID NO:20); (e) Gastrin G17 (SEQ ID NO:47); (f) Cat Ghrelin (SEQ ID NO:59); (g) Dog Ghrelin (SEQ ID NO:58); (h) HIV Env peptide 1 (SEQ ID NO:98); (i) HIV Env peptide 2 (SEQ ID NO:99); (j) CCR5PNt (SEQ ID NO:45); and (k) CCR5 ECL2 (SEQ ID NO:91).

In one preferred embodiment, the modified VLP of the invention is a mosaic VLP. In one further preferred embodiment, the mosaic VLP, in addition to the fusion protein of the invention, further comprises at least one protein, wherein the amino acid sequence of said protein is different from the fusion protein of the invention. In a further preferred embodiment of the invention, said protein is a coat protein of AP205. In a still further preferred embodiment, said protein is selected from the group consisting of: (a) SEQ ID NO:1; (b) SEQ ID NO:2; (c) SEQ ID NO:42; and (d) SEQ ID NO:67; (e) SEQ ID NO:68; (f) SEQ ID NO:69 and (g) a mutein of SEQ ID NO:1, or 67. The provision of coat protein of AP205, or muteins thereof, facilitates the assembly of the fusion protein of the invention into modified VLPs and stabilizes the formed modified VLPs. Various methods are available in the prior arts to express proteins with different sequences in one host cell, preferably in bacteria. One preferred method is to in-frame engineer a stop codon which allows suppression, such as an opal or an amber stop codon, at the 3′ of the nucleotide sequence encoding the first polypeptide. When the nucleotide sequence is expressed in a bacteria host, proteins with two different lengths will be generated. The coat protein of AP205 will be generated when the translation machinery recognizes the stop codon and stops the translation. The fusion of the invention will be produced when the translational machinery suppresses the stop codon and further translates the mRNA.

In one preferred embodiment, the modified VLP of the bacteriophage AP205 of the invention further comprises at least one immunostimulatory substance. Preferably the immunostimulatory substance is a Toll-like receptor ligand, preferably selected from the group consisting of: (a) immunostimulatory nucleic acids; (b) peptidoglycans; (c) lipopolysaccharides; (d) lipoteichonic acids; (e) imidazoquinoline compounds; (f) flagellines; (g) lipoproteins; (h) immunostimulatory organic molecules; (i) unmethylated CpG-containing oligonucleotides; and (j) any mixtures of substance of (a), (b), (c), (d), (e), (f), (g), (h) and (i). The inclusion of at least one immunostimulatory substance, preferably at least one Toll-like receptor ligand in the present inventive composition drastically increases the immunogenicity of the composition and enhances B and T cell responses. Therefore, the inventive compositions further comprising at least one immunostimulatory substance may be ideal vaccine compositions for prophylactic or therapeutic treatment against allergies, tumors and chronic viral diseases.

In another preferred embodiment, the immunostimulatory nucleic acid is preferably selected from the group consisting of: (a) a nucleic acid of bacterial origin; (b) a nucleic acid of viral origin; (c) a nucleic acid comprising unmethylated CpG motif; (d) a double-stranded RNA; (e) a single stranded RNA; and (g) a nucleic acid free of unmethylated CpG motif. Immunostimulatory nucleic acids that do not contain unmethylated CpG motif have been disclosed in the art, for example in WO 01/22972 which is incorporated herein by reference in its entirety. The term “nucleic acid,” as used herein, refers to a molecule composed of linearly covalently linked monomers (nucleotides). It indicates a molecular chain of nucleotides and does not refer to a specific length of the product. Thus, oligonucleotides are included within the definition of nucleic acid. The bond between the nucleotides is typically and preferably phosphodiester bond. Nucleic acids comprising modifications of bonds, for example, phosphorothioate bond, are also encompassed by the present invention.

In one preferred embodiment, the immunostimulatory nucleic acid is preferably selected from the group consisting of: (a) bacterial DNA that contains immunostimulatory sequences, in particular unmethylated CpG dinucleotides within flanking bases (referred to as CpG motifs) and (b) double-stranded RNA synthesized by various types of viruses. In one further preferred embodiment, the immunonucleic acid comprises or consists essentially of, or alternatively consists of double-stranded RNA poly I:C.

In one preferred embodiment, the unmethylated CpG-containing oligonucleotide comprises the sequence: 5′ X1X2CGX3X4 3′, wherein X1, X2, X3 and X4 are any nucleotide. Preferably, the oligonucleotide can comprise about preferably about 20 to about 300 nucleotides. In a preferred embodiment, the CpG-containing oligonucleotide contains one or more phosphorothioate modifications of the phosphate backbone. In an alternative preferred embodiment, the CpG-containing oligonucleotide is devoid of phosphorothioate modifications of the phosphate backbone. In one preferred embodiment, the unmethylated CpG-containing oligonucleotide comprises or consists of TCCATGACGTTCCTGAATAAT (SEQ ID NO:94).

In one further preferred embodiment, the unmethylated CpG-containing oligonucleotide comprises, or alternatively consists essentially of, or alternatively consists of a palindromic sequence. In a further preferred embodiment, said palindromic sequence is flanked by guanine nucleotides, preferably by at least 4 or 6, still more preferably by at least 8 or 10 guanine nucleotides. In one preferably embodiment, unmethylated CpG-containing oligonucleotide comprises or consists of GGGGTCAACGTTGAAGGGGGG (SEQ ID NO:95).

In one preferably embodiment, said palindromic sequence comprises, or alternatively consists essentially of, or alternatively consists of GACGATCGTC (SEQ ID NO: 70). In a very preferred embodiment, the unmethylated CpG-containing oligonucleotide comprises, or alternatively consists essentially of, or alternatively consists of the sequence GGGGGGGGGGGACGATCGTCGGGGGGGGGG (SEQ ID NO:71).

Other useful immunostimulatory nucleic acid sequences have been disclosed in the published WO2004/085635 and the disclosure is incorporated herein by way of reference. Detailed descriptions of Immunostimulatory substance, particularly immunostimulatory nucleic acid, more particularly oligonucleotides comprising unmethylated CpG have been disclosed in WO 03/024480, WO 03/024481 and PCT/EP/04/003165.

In one preferred embodiment, the immunostimulatory substance is mixed with the modified VLP. In another preferred embodiment, the immunostimulatory substance is bound to, preferably packaged inside, the modified VLP. Methods of mixing the immunostimulatory substances with the VLP-antigen have been disclosed in WO03/024480. Methods of packaging the immunostimulatory substances inside the VLP have been disclosed in WO 03/024481. The entire applications of WO 03/024480, 03/024481 and PCT/EP/04/003165 are therefore incorporated herein by way of reference. Furthermore, the packaged nucleic acids and CpGs, respectively, are protected from degradation, i.e., they are more stable. Moreover, non-specific activation of cells from the innate immune system is dramatically reduced

In one aspect, the invention provides a vaccine composition comprising the modified virus-like particle (VLP) of the invention, preferably the vaccine composition further comprises a buffer. In one embodiment, the vaccine composition further comprises an adjuvant. The administration of the at least one adjuvant may hereby occur prior to, contemporaneously or after the administration of the inventive composition. Adjuvants either facilitate targeting of dendritic cells, contain substances that activate dendritic cells or induce the formation of a local antigen depot. Examples of the at least one adjuvant include and preferably consist of complete and incomplete Freund's adjuvant, aluminum hydroxide, aluminium salts, and modified muramyldipeptide. Further adjuvants are mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. Still further adjuvant include immunostimulatory nucleic acid, preferably the immunostimulatory nucleic acid contains one or more modifications in the backbone, preferably phosphorothioate modifications. The modification is to stabilize the nucleic acid against degradation.

The adjuvants can also comprise a mixture of these substances. However, the term “adjuvant”, as used within the context of this application, refers to an adjuvant not being the modified VLP of the present invention, and not being, if applicable, the immunostimulatory substance, preferably immunostimulatory nucleic acid, packaged inside the modified VLP, rather in addition to said modified VLP and in addition to, if applicable, the immunostimulatory substance, preferably immunostimulatory nucleic acid, packaged inside the modified VLP.

In one preferred embodiment, the vaccine composition is devoid of adjuvant. Thus, the administration of the vaccine composition of the invention to a patient will preferably occur without administering at least one adjuvant to the same patient prior to, contemporaneously or after the administration of the vaccine. An advantageous feature of the present invention is the high immunogenicity of the composition, even in the absence of adjuvants. The absence of an adjuvant, furthermore, minimizes the occurrence of unwanted inflammatory T-cell responses representing a safety concern in the vaccination, in particular in the vaccination against self antigens.

The invention further discloses a method of immunization comprising administering the vaccine of the present invention to an animal or a human. The animal is preferably a mammal, such as cat, sheep, pig, horse, bovine, dog, rat, mouse and particularly human. The vaccine may be administered to an animal or a human by various methods known in the art, but will normally be administered by injection, infusion, inhalation, oral administration, or other suitable physical methods. The conjugates may alternatively be administered intramuscularly, intravenously, transmucosally, transdermally, intranasally, intraperitoneally or subcutaneously. Components of conjugates for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

The nature of the immune response can be affected by the nature of the antigen, route of introduction into the body, dose and dosage regimen, repetitive nature of the antigen, host background, or signalling factors of the immune system. An immune response may be tailored by the application of both art known theory and routine experimentation. Vaccines of the invention are said to be “pharmacologically acceptable” if their administration can be tolerated by a recipient individual. Further, the vaccines of the invention will be administered in a “therapeutically effective amount” (i.e., an amount that produces a desired physiological effect). The nature or type of immune response is not a limiting factor of this disclosure.

In another aspect, the invention provides a pharmaceutical composition comprising: (a) the modified VLP of the invention; and (b) an acceptable pharmaceutical carrier. When vaccine of the invention is administered to an individual, it may be in a form which contains salts, buffers, adjuvants, or other substances which are desirable for improving the efficacy of the conjugate. Examples of materials suitable for use in preparation of pharmaceutical compositions are provided in numerous sources including REMINGTON'S PHARMACEUTICAL SCIENCES (Osol, A, ed., Mack Publishing Co., (1990)).

In one further aspect, the invention provides a method of treating or preventing a disease, disorder or physiologic conditions in an individual, wherein said method comprising administering to an animal or a human the modified VLP of the invention, the vaccine composition of the invention or the pharmaceutical composition of the invention. In another aspect, the invention provides a use of the modified VLP for the manufacturing of a medicament for the treatment or prevention of a disease, a disorder or physiologic conditions in an animal or in human.

In one aspect, the invention provides a method for producing the modified VLP of the invention, comprising the steps of: (a) (optional) in-frame ligating a nucleotide sequence encoding a spacer with either the first nucleotide sequence encoding the first polypeptide or the second nucleotide sequence encoding the second polypeptide; (b) in-frame ligating said second nucleotide sequence with said first nucleotide sequence, resulting in a third nucleotide sequence encoding said fusion protein; (c) (optional) introducing a stop codon which allows suppression at the 3′ of the first nucleotide sequence; (d) expressing said third nucleotide sequence in a host, preferably under the condition that the resulting expressed proteins are capable of forming said modified VLPs; (e) purifying said modified VLPs obtained from step (d).

In one aspect, the invention provides a fusion protein comprising a polypeptide, wherein said polypeptide is fused to either the N- or C-terminus, or to both terminus, of a coat protein, or a mutein thereof, of AP205 bacteriophage, wherein preferably said polypeptide consists of 1-60 amino acids, preferably consists of 3-40, more preferably 5-30, still more preferably 10-25 amino acids, still more preferably 1-15, still more preferably 3-15, more preferably 1-11, more preferably 3-11, more preferably 1-8, more preferably 3-8 amino acids; and wherein said fusion protein is capable of forming a VLP.

In another preferred embodiment, the polypeptide to be fused to the terminus of the coat protein, or muteins thereof, of AP205 bacteriophage, has less than 30 amino acids, preferably less than 20 amino acids, more preferably less than 15 amino acids, even more preferably less than 10 amino acids.

In one further preferred embodiment, the polypeptide is fused to the N- or C- or to both terminus of the coat protein, or a mutein thereof, of AP205 selected from the group consisting of: (a) SEQ ID NO:1; (b) SEQ ID NO:2; (c) SEQ ID NO:42; (d) SEQ ID NO:67, (e) SEQ ID NO:689; (f) SEQ ID NO:69 and (g) a mutein of SEQ ID NO:1 or SEQ ID NO:67.

In one further aspect, the invention provides a nucleotide sequence encoding the fusion protein of the invention. One amino acid sequence of a mutein maybe encoded by more than one nucleotide sequences due to the degeneracy of the genetic code. Thus all the nucleotide sequences that encode the same amino acid sequence of a mutein are encompassed by the present invention.

### EXAMPLES

### Example 1

### Construction of Plasmids for Fusing Antigens to the N- and C-Terminus of AP205 Coat Protein

When referring to the N-terminus of AP205 coat protein in the cloning work described below, the term “N-terminus” refers to the first Alanine, not to the initial Methionine.

Construct 378-2: addition of a short GSGG spacer and NcoI and Kpn2I cloning sites within the nucleic acid sequence coding for the spacer at the N-terminus of the AP205 coat protein.

This construction was made by PCR using pAP283-58 (SEQ ID NO:3) as template, and using an upstream primer p2.561 (SEQ ID NO:4) containing a NcoI− and a downstream primer p1.46 (SEQ ID NO:5) containing a HindIII− restriction site. The PCR fragment was digested with NcoI and HindIII and cloned in the same restriction sites into a pQb185, resulting in plasmid pAP378-2.

Construct 382-2: addition of a long GSGTAGGGSGS spacer and NcoI and Kpn2I cloning sites within the nucleic acid sequence coding for said spacer at the N-terminus of AP205 coat protein by PCR.

This construction was made by PCR using 378-2 as a template and using an upstream primer p2.589 (SEQ ID NO:6) containing NcoI and a downstream primer p1.46 (SEQ ID NO:5) containing HindIII restriction sites. The PCR fragment was digested with NcoI and HindIII and cloned in the same restriction sites into pQb185, resulting in plasmid pAP382-2.

Construct: 409-44: Addition of a short GSG spacer and Kpn2I and Mph1103I cloning sites within the nucleic acid sequence coding for said spacer at the C-terminus of AP205 coat protein.

This construction was made by PCR with plasmid pAP283-58 (SEQ ID NO:3) as template using an upstream primer p1.45 (SEQ ID NO:7) containing XbaI and a downstream primer p2.587 (SEQ ID NO:8) containing Mph1103I restriction sites. The PCR fragment was digested with XbaI and Mph1103I and cloned in the same restriction sites into a pQb10, resulting in plasmid pAP409-44.

Construct 405-61: addition of a long GTAGGGSG spacer and Kpn2I and Mph1103I cloning sites within the nucleic acid coding for it at the C-terminus of AP205 coat protein.

This construction was made by PCR with 409-44 as template using an upstream primer p1.45 (SEQ ID NO:7) containing XbaI and a downstream primer p2.588 (SEQ ID NO: 9) containing Mph1103I restriction sites. The PCR fragment was digested with XbaI and Mph1103I and cloned in the same restriction sites into a pQb10, resulting in plasmid pAP405-61.

Constructs 378-2, 382-2, 409-44, 405-61 and their corresponding plasmids are referred to as 378, (pAP378), 382 (pAP382), 409 (pAP409) and 405 (pAP405) thereafter for the sake of simplicity. In the following examples, various antigens have been cloned into the above described vectors.

In order to test the effect of a linker on particle assembly, protein from construct 378 were expressed as describe in EXAMPLE 2, and the assembly to a VLP was demonstrated by EM and immunodiffusion (Ouchterlony) assays.

### Example 2

### Expression of AP205 Fusion Proteins

E. coli JM109 cells were transformed with the corresponding AP205 fusion protein plasmid. A seed culture was prepared by inoculated an individual colony grown on agar containing 100 mg/l Ampicillin into LB medium containing 20 mg/l Ampicillin and growing the culture overnight at 37° C. without shaking. For expression, the overnight culture was diluted at 1:50 in M9 medium supplemented with casaminoacids (Difco) and containing 20 mg/l Ampicillin and growth of the culture carried out at 37° C. with vigorous aeration for 14-20 hours. Cells were collected at 6000 rpm for 15′-20′ at 4-8° C.

### Example 3

### Cloning, Expression and Purification of the Modified VLP Comprising Fusion Proteins of the Coat Protein Fused with the D2 Peptide

Cloning of the D2 peptide at the C-terminus of the AP205 coat protein

The DNA fragment coding for the D2 peptide (TSNGSNPSTSYGFAN, SEQ ID NO:10) was created by annealing two oligonucleotides—oligo2.196 (SEQ ID NO:11) and oligo 2.197 (SEQ ID NO:12). The obtained fragment was digested with Kpn2I and Mph1103I and cloned in the same restriction sites into pAP409-44 and pAP405-61 under the control of E. coli tryptophan operon promoter. The resulting constructs are:

| | |
|---|---|
| 418-7 (based on 409-44): | AP205 coat protein- |
| | GSG-D2 peptide |
| 420-21 (based on 405-61): | AP205 coat protein- |
| | GTAGGGSG-D2 peptide. |

### Cloning of the D2 Peptide at the N-Terminus of AP205 Coat Protein

The fragment coding for the D2 peptide (TSNGSNPSTSYGFAN, SEQ ID NO:10) was created by annealing two oligonucleotides—oligo2.590 (SEQ ID NO:13) and oligo 2.591 (SEQ ID NO:14). The obtained fragment was digested with NcoI and Kpn2I and cloned in the same restriction sites into the vectors pAP378-2 and pAP382-2.

The resulting construct are:

421-8 (based on 378-2): MG—D2 peptide—GSGG—AP205 coat protein. As a result of the cloning procedure amino acid 14 of SEQ ID NO:1 was changed to aspartate.
422-2 (based on 382-2): MG—D2 peptide—GSGTAGGGSGS—AP205 coat protein. Constructs 418-7, 420-21, 421-8 and 422-2 are referred to as 418, 420, 421 and 422 thereafter for the sake of simplicity

### Purification

Standard buffer for all the steps of purification was the NET buffer: 20 mM Tris-HCl, pH 7.8 with 5 mM EDTA and 150 mM NaCl.

Cell lysates were purified over a CL-4B column, and the pooled eluted fractions were further purified by CsCl gradient ultracentrifugation. Concentrations of the purified proteins were determined by the Bradford test.

Display of the antigen was tested in an inhibition ELISA, where the peptide D2 was conjugated to RNase via an amino acid spacer (CGG) and the cross-linker SPDP and coated on an ELISA plate, while the VLPs displaying the D2 peptide were incubated with an anti-D2 rabbit antiserum raised against a D2-Fr fusion protein assembled into a VLP. Detection was performed with a donkey anti-rabbit HRP conjugate.

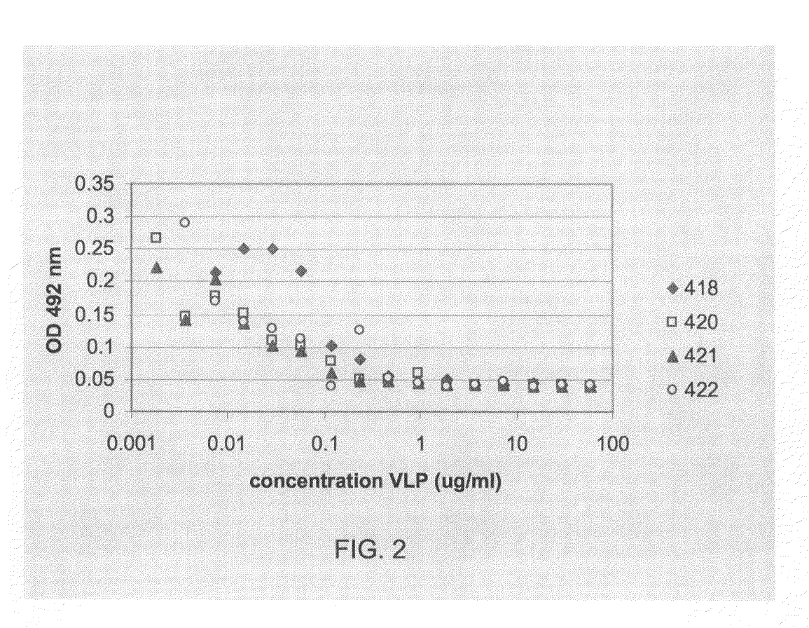

All four modified VLPs (D2 peptide at either the N- or the C-terminus with either the short or the long spacers) inhibited the binding of the anti-D2 antiserum to the D2 peptide conjugated to RNase and coated on the plate as shown by ELISA, indicating the display of the D2 peptide on the modified VLPs assembled from the four fusion proteins (FIG. 2).

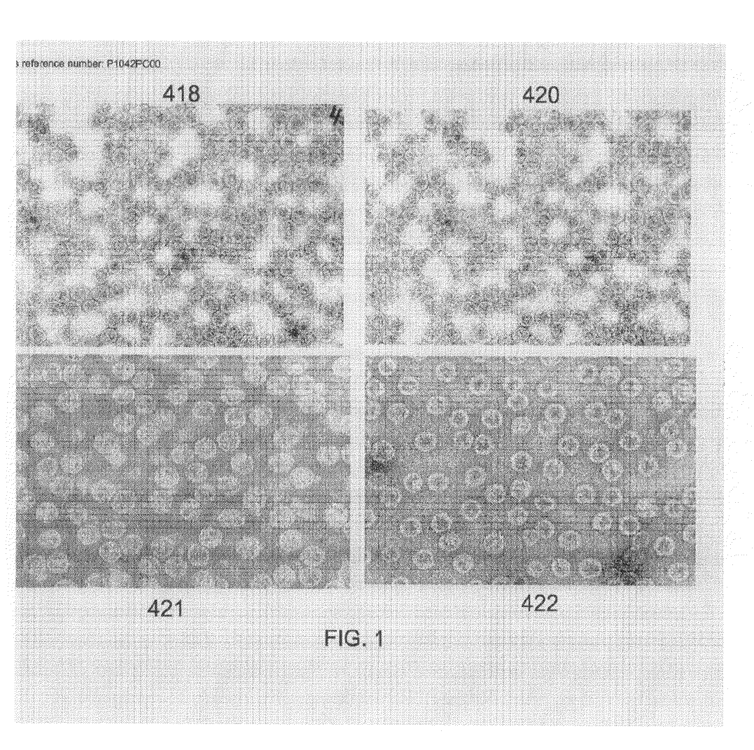

Furthermore, electronmicrographs of all four modified VLP purified by gel filtration confirmed capsid assembly (FIG. 1).

### Example 4

### Immunization of Mice and Analysis of the Immune Response with the Modified VLPs of AP205 Displaying the D2 Peptide

Mice (n=3 per group) were immunized subcutaneously on day 0 and 14 with 25 μg proteins from constructs 418-7, 420-21, 421-8 and 422-2, which are thereafter referred to as 418, 420, 421 and 422. The proteins were diluted to a final volume of 200 μl in PBS, and 100 μl were injected in the left and right inguinal region of each animal. Animals were bled on day 14 and 21, and the antibody response was measured in an ELISA. Briefly, a variant of D2 peptide containing the amino acid sequence CGG at its N-terminus was coupled to RNase using the cross-linker SPDP. The resulting conjugate was coated overnight at 4° C. Binding of the sera was detected with a Horseradish-peroxidase goat anti-mouse IgG conjugate.

All four modified VLPs elicited high titer antibody responses against the D2 peptide, while no binding of sera was detected with pre-immune serum, showing the specificity of the binding. The titers were measured as the dilution giving half-maximal binding, and the average titer of the three animals was 10700±8600 for construct 418, 1:10200±3000 for construct 420, 1:7900±5500 for construct 421, and 1:2018±2500 for construct 422.

### Example 5

### Cloning, Expression and Purification of the Modified VLP of AP205 Displaying the Angio I Peptide

### Cloning of the Angio I Peptide at the C-Terminus of AP205 Coat Protein

The fragment coding for the Angio I peptide (DRVYIHPF, SEQ ID NO:15) was created by annealing two oligonucleotides—oligo3.216 (SEQ ID NO:16) and oligo 3.217 (SEQ ID NO:17). The obtained fragment was digested with Kpn2I and Mph1103I and cloned in the same restriction sites into the vectors pAP409-44 and pAP405-61 under the control of E. coli tryptophan operon promoter.

The new constructs are:

| | |
|---|---|
| 441-9 (based on 409-44): | AP205 coat protein- |
| | GSG-DRVYIHPF |
| 442-7 (based on 405-61): | AP205 coat protein- |
| | GTAGGGSG-DRVYIHPF. |

### Cloning of the Angio I Peptide at the N-Terminus of AP205 Coat Protein

A peptide with amino acid sequence DRVYIHPF (SEQ ID NO:15) is referred herein as Angio I peptide. The fragment coding for the Angio I peptide was created by annealing two oligonucleotides—oligo3.218 (SEQ ID NO:18) and oligo 3.219 (SEQ ID NO:19). The obtained fragment was digested with NcoI and Kpn2I and cloned in the same restriction sites into the vectors pAP378-2 and pAP382-2.

The new constructs are:

| | |
|---|---|
| 446-6 (based on 378-2: | MG-DRVYIHPF--GSGG-AP205 |
| | coat protein |
| 447-9 (based on 382-2): | MG-DRVYIHPF--GSGTAGGGSG |
| | S-AP205 coat protein |

Constructs 441-9, 442-7, 446-6 and 447-9 are referred to as 441, 442, 446 and 447 thereafter for the sake of simplicity.

### Purification

Cells were lysed by three times freeze thaw cycles in a Tris-buffered lysis buffer containing 1 mg/ml lysozyme and 0.1% Tween 20 followed by ultrasonication. The lysate was clarified by centrifugation yielding lysate 1, and the pellet reextracted with lysis buffer yielding lysate 2. The supernatants were thereafter purified further by a combination of gel filtration steps, and the resulting pure fractions were combined.

The combination of gel filtration steps for each construct are described in the following:

Construct 441: lysate 1 was loaded first on a Sepharose CL-2B, then on a Sepharose 6B column. The second lysate was loaded first on a CL-4B, then on a CL-2B column.
Construct 442: lysate 1 was first loaded on a CL-2B, then on a CL-4B and finally on a sepharose 6B column. Lysate 2 was first loaded on a CL-4B, then on a sepharose 6B column.
Construct 446: lysate 1 was purified over a CL-2B followed by a sepharose 6B column. Lysate 2 was discarded.
Construct 447: Lysate 1 was purified over a CL-4B followed by a sepharose 6B column. Lysate 2 was purified twice over a CL-4B column.

All four constructs formed modified VLPs as confirmed by electron microscopy. Display of the Angio I peptide on the VLPs was further confirmed by ELISA, whereby the VLPs were coated at a concentration of about 10 μg/ml, and the binding of two antisera raised in mice against the Angio I or Angio XVIII peptide, respectively, was assessed. All four modified VLPs were positive in the ELISA, confirming the display of the Angio I peptide on the modified VLPs.

### Example 6

### Cloning, Expression and Purification of the Modified VLP of AP205 Displaying GnRH and Immunization of Mice

### Cloning of GnRH at the C-Terminus of AP205 Coat Protein

The DNA fragment coding for the GnRH peptide (EHWSYGLRPG, SEQ ID NO:20) was created by annealing two oligonucleotides—oligo 4.56 (SEQ ID NO:21) and oligo 4.57 (SEQ ID NO:22). The obtained fragment was digested with Kpn2I and Mph1103I and cloned in the same restriction sites into the vector pAP405-61 under the control of E. coli tryptophan operon promoter.

The resulting construct was:

489-7 (based on 405-61): AP205 coat protein—GTAGGGSG—EHWSYGLRPG, this construct is referred as construct 489 for the sake of simplicity.

### Purification

Cells were lysed as described in EXAMPLE 4. The pellet was extracted with four portions of a buffer containing 7 M urea and 0.05 M Tris. The pooled supernatants were loaded on a Sepharose CL-2B column equilibrated in NET buffer, and rechromatographed on a sepharose 6B column. Capsid assembly was confirmed by EM analysis.

### Immunization of Mice with Modified VLP Comprising Fusion of AP205 Coat Protein and GnRH and Analysis of the Immune Response

Mice (n=5 per group) were immunized subcutaneously on day 0 with 50 μg of protein expressed from construct 489. The protein was diluted to a final volume of 200 μl with 20 mM Hepes pH7.2, and 100 μl were injected in the left and right inguinal region of each animal. Animals were bled on day 21, and the antibody response was measured in an ELISA. Briefly, a variant GnRH peptide containing the amino acid sequence CGG at its N-terminus was coupled to RNAse using the cross-linker SPDP. The resulting conjugate was coated overnight at 4° C. Binding of the sera was detected with a Horseradish-peroxidase goat anti-mouse IgG conjugate.

The protein from construct 489 elicited high titer antibody responses against the GnRH peptide, while no binding of sera was detected with pre-immune serum, showing the specificity of the binding. The titers were measured as the dilution giving half-maximal binding, and the average titer of the five animals was 1:18329 with standard derivation of 9245.

### Example 7

### Cloning, Expression and Purification of AP205 VLP Displaying the Nef55 Epitope Cloning

Nef55 (SEQ ID NO:23) is a polyepitope derived from an HIV Nef consensus sequence, and selected to contain the highest possible number of T-cell epitopes and for solubility. Nef55 was amplified by PCR from the DNA encoding another polyepitope of HIV Nef, the Nef74 polyepitope. The DNA coding for Nef55 was assembled from two fragments generated by PCR encoding amino acid sequences GVGFPVRPQVPLRPMTYKAAV-DLSHFLKEKGGLE and GPGIRYPLTFGWCFKLVPVEP. For the amplification of the 34 amino acid fragment an upstream primer p3.242 (SEQ ID NO:24) containing Kpn2I restriction site and a downstream primer p3.222 (SEQ ID NO:25) were used. For the amplification of the 21 amino acids fragment an upstream primer p3.223 (SEQ ID NO:26) and a downstream primer p3.225 (SEQ ID NO:27) containing Mph1103I restriction site were used. The fragment fusion was realized using assembly PCR with the same upstream and downstream primers as above. The obtained fragment was digested with Kpn2I and Mph1103I and cloned in the same restriction sites into the vectors pAP409-44 and pAP405-61 under the control of E. coli tryptophan operon promoter.

The resulting constructs were:

| | | |
|---|---|---|
| Construct 457-17 | (based on 409-44): | AP205 coat |
| | | protein-GSG- |
| | | Nef55 |
| Construct 459-35 | (based on 405-61): | AP205 coat |
| | | protein-GTAGGGS |
| | | G-Nef55 |

Constructs 457-17 and 459-35 are referred to hereinafter as 457 and 459, respectively, for the sake of simplicity.

### Purification

Cells were lysed as described in Example 4. For construct 457, pooled lysates were purified by sucrose gradient ultracentrifugation followed by a Sepharose 2B column. Although capsids were visible in the EM analysis of the partially purified protein, they were of poor quality with a lot of half capsids being visible.

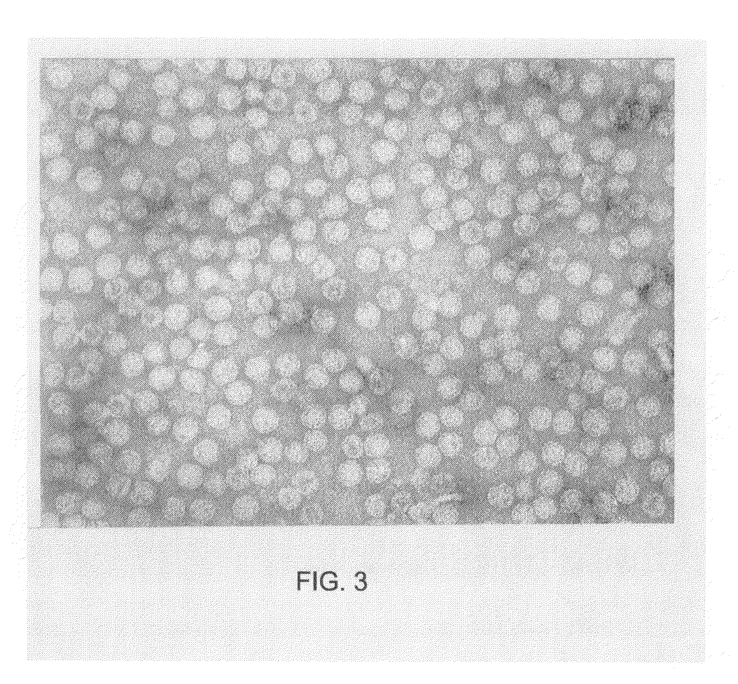

For construct 459, lysate 2 was loaded on a Sepharose 2B column (size 2.5×45 cm) eluted at 2 ml/h, concentrated on Amicon centrifugal concentrators, and loaded on a Sephadex 2 B column. The protein was further purified twice by CsCl gradient ultracentrifugation. Assembly into VLPs of the fusion protein was confirmed by EM analysis, which showed regularly nicely shaped capsids (FIG. 3).

HHD mice express a chimeric monochain class I molecule with a human β2-microglobulin covalently linked to the N-terminus of A2 α1 and α2 domains fused with Db α3 domain (Firat, H. et al 1999, Eur. J. Immunol., 29:3112). The HLA-A2 transgene expression in these mice allows investigating the capacity of AP205-Nef55 VLPs to prime CTL in vivo. Furthermore, the effect of adjuvants, as ISS can be studied in vivo.

HHD mice are either left untreated or immunized by injecting subcutaneously 100 μg AP205-Nef55. Eight days later spleenocytes are isolated and T-cell induction is analyzed in an intracellular cytokine staining assay for interferon-gamma in proliferation assays (for Th cell response, Belshe R. B. et al., J. Inf. Dis. 183: 1343-1352 (2001)), in ELISPOT assays (Oxenius, A. et al., Proc. Natl. Acad. Sci. USA 99: 13747-13752 (2002)), or in Cytotoxicity assays (Belshe R. B. et al., J. Inf. Dis. 183: 1343-1352 (2001)) using appropriate HLA-A2 restricted T-cell epitopes for stimulation which can be identified for example using the online database on HIV epitopes and consensus sequence, http://hiv-web.lanl.gov/seq-db.html.

### Example 8

### Cloning, Expression and Purification of the Modified VLP of AP205 where the Opal Codon Separates the Nef55 from the C-Terminus of AP205 Coat Protein and Resulting in Mosaic VLP Cloning

The construction 459-35 was used as the source of AP205 coat protein and Nef55 coding sequences. The new construction 512 was designed by two-step PCR, in order to introduce the opal codon between the sequence coding for the coat protein and the one coding for the amino acid spacer GTAGGGSG.

The opal codon introduction was realized using inverse PCR. The inverse primers were designed in inverted tail-to-tail directions and with a TGA insertion using primers p4.101 (SEQ ID NO:28) and p4.102 (SEQ ID NO:29). An upstream primer p1.44 (SEQ ID NO:30) containing NcoI restriction site and a downstream primer p74-2 (SEQ ID NO:31) complementary to the non coding region 23 nucleotides downstream of the C-terminus of Nef56 peptide in the construction 459-35 were used.

The PCR fragment was digested with Nco I and Hind III and cloned in the same restriction sites into a pGEM-derived expression vector under the control of E. Coli tryptophan operon promoter, resulting in plasmid pAP512-24.

The resulting construction is:

Construct 512-24: AP205 coat protein—opal codon—GTAGGGSG—Nef55, which is thereafter referred to as 512 for the sake of simplicity.

### Expression

E. coli JM109 cells containing helper plasmid pISM3001 were transformed with plasmid pAP512-24 and plated on LB agar containing 100 mg/l Ampicillin and 10 mg/L Chloramphenicol. Subsequent steps were performed as described above, except that 10 mg/L Chloramphenicol was added to all culture media.

### Purification

Cells were lysed as described in Example 4. Lysate 1 was purified over a Sepharose 4B column (1.2×25 cm) eluted at 1 ml/h with a Tris, NaCl, EDTA buffer (NET buffer). Eluted fractions were pooled, concentrated on a Amicon centrifugal concentrators, and loaded on a Sepharose 6 Bcolumn (1.2×35 cm), eluted at 2 ml/h. Expression of both the AP205 coat protein and the fusion protein was confirmed by western blot analysis of the purified VLP and capsid assemble was confirmed by EM analysis.

### Example 9

### Cloning, Expression and Purification of the Modified VLP of AP205 Displaying the Extended p33 Peptide at the N-Terminus of AP205 Coat Protein Cloning

The fragment coding for the extended p33 peptide (AKSLKAVYNFATMA, SEQ ID NO:32) was created by annealing two oligonucleotides—oligo3.309 (SEQ ID NO:33) and oligo 3.310 (SEQ ID NO:34). The obtained fragment was digested with NcoI and Kpn2I and cloned in the same restriction sites into the vectors 378-2 and 382-2 under the control of E. coli tryptophan operon promoter.

The resulting construct is:

Construct 466 (based on: 382-2): MAKSLKAVYNFATMA—GSGTAGGGSGS—AP205 coat protein. The extended p33 peptide contains the CTL epitope KAVYNFATM.

### Purification

Cells were lysed as described in Example 4. The pellet isolated from lysate 2 was additionally extracted with a buffered 7M Urea, pH 7.5 Tris buffer. The modified VLPs displaying the p33 peptide were purified over a Sepharose 4B column (1.2×25 cm), equilibrated in NET buffer and eluted at 1 ml/h. Capsid assembly was confirmed by the elution volume from the column which has been calibrated with VLP.

### Example 10

### Cloning, Expression and Purification of fr Coat Protein Fused to the p33 Peptide

The sequence of the extended p33 peptide (KSLKAVYNFATMA, SEQ ID NO:32) contains the p33 CTL epitope (KAVYNFATM).

The following oligonucleotides were synthesized:

(SEQ ID NO:39)

5′ CG AAA TCT CTT AAA GCG GTT TAG AAC TTC GCT ACC
ATG GCT T.

(SEQ ID NO:40)

5′ CGA AGC CAT GGT AGC GAA GTT GTA AAC CGC TTT AAG
AGA TTT

Oligonucleotides 1 contains a unique Nco I site to facilitate the selection of clones. The oligonucleotides were treated with T4 Polynucleotide kinase for 30 min at 37° C., the mix 1,2 was subsequently heated to 100° C. for 3 min and slowly cooled to room temperature.

### Vector Preparation

The plasmid pFRd8 was cleaved at the AsuII site for 3 h at 37° C. The vector fragment was purified, and ligated to the annealed oligo 1 and 2. The resulting construct had the sequence KSLKAVYNFATMA inserted between amino acid 2 and 3 of fr coat protein (initial Alanine after cleaved N-terminal alanine is position 1).

### Preparation of Cell Extract for Protein Purification

The initial steps of protein purification, including the preparation and sonication of bacterial cells lysates, were performed with buffer A.

Buffer A: 250 mM NaCl, 50 mM Tris HCl pH 7.2, 5% Glycerol, 2 mM EDTA and Lysozyme added to 20 μg/ml.

1 g cell was resuspended in three volume of buffer A and the suspension was incubated at 4° C. for 20 min, then the suspension was sonicated at 200 watt-seconds for three 30 s bursts. The sonicated suspension was incubated at 110° C. for 20 min, at which time an equal volume of the same buffer was added along with PMSF 1 mM. The mixture was sonicated as before, and then centrifuged at 10,000×g for 30 min. The pellet was extracted with 3 ml of 4M urea.

Polyimin P (10% w/v pH7.2) was added slowly to the supernatant to the final concentration of 0.35% w/v and the turbid solution was centrifuged at 6,000 g for 15 min. The supernatant was precipitated with ammonium sulphate to 35% saturation, the solution stirred for additional 3 h, and then centrifuged at 8,000×g for 15 min. The pellet was resuspended in 1 cell volume (1 ml) of buffer B.

Buffer B: 1M NaCl, 10 mM Tris HCl pH 7.2, 5% Glycerol, 1 mM EDTA.

The supernatant was subsequently precipitated with ammonium sulphate to 50% saturation. The aliquots from each step of protein preparation were applied to SDS PAGE Electrophoresis and subjected to Western Blot analysis. The sample was used for future purification in Column chromatography and sucrose gradient centrifugation.

The protein preparation obtained by ammonium sulphate precipitation at 35% saturation in buffer B was analysed by EM, but no capsids could be detected. Nevertheless, an analytical amount of the proteins was purified on Sephacryl S-200 or Sephacryl S-400 gel filtration column for comparison. The protein was eluted with a buffer containing Tris-HCl pH7.2, 0.5M NaCl, 1 mM EDTA. The collected 12 fractions were analysed by SDS-PAGE. The fractions that correspond to the peak of the sephacryl 400 run were collected and analysed by EM (Electron Microscopy) analysis. The electron microscopy analysis did not detect any particles in the purified fractions, showing that fusion of the p33 peptide to fr coat protein prevented capsid assembly.

### Example 11

### Fusing Various Antigens to the Coat Protein of AP205

Four plasmids for fusing antigens either to the N- (Construct 378-2 and 382-2) or the C-terminus (construct 409-44 and 405-61) of the coat protein of AP205 with either long spacer or short spacer are obtained from EXAMPLE 1. The four vectors contain unique restriction sites into which the sequence to be fused to AP205 can be inserted. Briefly, two complementary oligonucleotides which contain the restriction sites present in the respective vector and encode the desired amino acid sequence (see below) to be fused in frame with the AP205 coding sequence are synthesized. A stop codon is also included at the end of the coding sequence when fusion is effected at the C-terminus of AP205. The two oligonucleotides are then annealed and digested with the appropriate restriction enzymes and cloned into the respective AP205 fusion expression vector.

Nucleotide sequence encoding CCR5 extracellular domain fragment ECL2A (SEQ ID NO:46, RSQKEGLHYT) is in-frame ligated into all four plasmids.

Nucleotide sequence encoding CXCR4 176-185 (SEQ ID NO:49) is in-frame ligated into all four plasmids.

Nucleotide sequence encoding human C5a fragment 55-74 (SEQ ID NO:46) is in-frame ligated into all four plasmids.

Nucleotide sequence encoding gastrin G17 (SEQ ID NO:47, EGPWLEEEEEAYGWMDF) is in-frame ligated into all four plasmids.

Nucleotide sequence encoding CETP fragment 461-476 (SEQ ID NO:51) is in-frame ligated into all four plasmids.

Nucleotide sequence encoding Bradykinin (SEQ ID NO:52) is in-frame ligated into all four plasmids.

Nucleotide sequence encoding des-Arg-Bradykinin (SEQ ID NO:53) is in-frame ligated into all four plasmids.

The expression and purification of the above fusion proteins are substantially the same as described in EXAMPLE 2 and 3. To check the formation of VLPs with the AP205 fusion proteins the samples are analysed by electron microscopy.

### Example 12

### Immunization of Mice with VLPs of AP205 Coat Protein Fused with Various Antigens

Mice (n=3 per group) are immunized subcutaneously on day 0 and 14 with 25 μg proteins of VLPs of AP205 fusion proteins obtained from EXAMPLE 11. The proteins are diluted to a final volume of 200 μl in PBS, and 100 μl are injected in the left and right inguinal region of each animal. Animals are bled on day 14 and 21, and the antibody response is measured in an ELISA.

Briefly, the antigen to-be-tested is conjugated to RNase via an amino acid spacer (CGG) and the cross-linker SPDP and coated on an ELISA plate overnight at 4° C. Binding of the sera is detected with a Horseradish-peroxidase goat anti-mouse IgG conjugate.

### Example 13

### Immunization of Pigs with VLPs AP205 Coat Protein Fused with GnRH

Pigs (n=2 per group) were immunized subcutaneously on day 0 with 400 μg of protein expressed and purified from construct 489 as described in EXAMPLE 6. The protein was diluted to a final volume of 1 mL with 5 mM Phosphate/100 mM NaCl buffer, pH6.8, containing a final concentration of 15% DEAE Dextran as an adjuvant. The vaccine was injected subcutanouesly behind the ear of each animal. Control animals (n=2 per group) were immunized with 1 mL Qβ VLP (0.4 mg/mL), prepared in 20 mM Hepes buffer pH7.2 and containing 15% DEAE Dextran. Animals were boosted on day 28 with the same amount of vaccine compositions that were used for the initial immunization. Animals were bled on day 28 and 49, and the antibody response was measured in an ELISA. Briefly, a variant GnRH peptide containing the amino acid sequence CGG at its N-terminus was coupled to RNAse using the cross-linker SPDP. The resulting conjugate was coated overnight at 4° C. Binding of the sera was detected with a Horseradish-peroxidase rabbit anti-swine IgG conjugate.

As shown in TABLE 1, pigs immunized with AP205 coat protein fused to GnRH elicited high titer antibody responses against the GnRH peptide, while no binding of sera was detected with pre-immune serum, nor with the serum of Qβ immunized control pigs, showing the specificity of the binding. The titers were measured as the dilution giving half-maximal binding.

TABLE 1

| anti-GnRH IgG titer | d28 | d49 |
|---|---|---|
| AP205-GnRH #1 | 1:857 | 1:1344 |
| AP205-GnRH #2 | 1:140 | 1:603 |
| Qb #1 | 1:20 | 1:25 |
| Qb #2 | 1:21 | 1:24 |

### Example 14

### Cloning, Expression and Purification of the Modified VLP Comprising Fusion Protein of the Coat Protein and the preS1 (aa21-47) Peptide

### Cloning of the preS1 Peptide at the C-Terminus of the AP205 Coat Protein

The DNA fragment coding for the preS1 peptide (PLGFFPDHQLDPAFRANTANPDWDFNP, SEQ ID NO:62) is created by annealing two oligonucleotides—oligo preS1-1 (SEQ ID NO:63) and oligo preS1-2 (SEQ ID NO:64). The obtained fragment is digested with Kpn2I and Mph1103I and cloned in the same restriction sites into pAP409-44 and pAP405-61 under the control of E. coli tryptophan operon promoter. The resulting constructs are:

| | | |
|---|---|---|
| preS1-A | (based on 409-44): | AP205 coat protein-GSG- |
| | | preS1 peptide |
| preS1-B | (based on 405-61): | AP205 coat protein-GTAGG |
| | | GSG-preS1-peptide. |

### Cloning of the preS1 Peptide at the N-Terminus of AP205 Coat Protein

The fragment coding for the preS1 peptide is created by annealing two oligonucleotides—preS1-3 (SEQ ID NO:65) and oligo preS1-4 (SEQ ID NO:66). The obtained fragment is digested with NcoI and Kpn2I and cloned in the same restriction sites into the vectors pAP378-2 and pAP382-2.

The resulting construct are:

| | | |
|---|---|---|
| preS1-C | (based on 378-2): | MG-preS1 peptide-GSGG- |
| | | AP205 coat protein. |
| preS1-D | (based on 382-2): | MG-preS1 peptide-GSGTAGG |
| | | GSGS-AP205 coat |
| protein. | | |

Expression and purification of the above mentioned fusion proteins are carried out substantially the same as described for the AP205-D2 fusions.

### Example 15

### Generation of PreS1 (aa21-47)-specific antibodies and determination of neutralizing activity

Adult male, C57BL/6 mice (5 per group) are vaccinated with the AP205-preS1 (aa21-47) VLPs or, as a control, with AP205 VLP. For each mouse, 100 μg of dialyzed vaccine is diluted in PBS to a volume of 2001 and injected subcutaneously (1001 on two ventral sides) on days 0 and 14. The vaccine is administered without adjuvant. As a control, a group of mice is injected with PBS. Mice are bled out by heart puncture on day 21 and serum is purified. Sera from the 5 mice in each group are pooled and centrifuged for five minutes at 14'000 rpm. The supernatant is loaded on a column of 3 ml prewashed protein G sepharose (Amersham Biosciences). The column is then washed with 10 column volumes of PBS and eluted with 100 mM glycine pH2.8. 1 ml fractions are collected in tubes containing 200 μl 1M Tris pH8.0. The protein containing fractions are pooled and concentrated using a Millipore Ultrafree centrifugal filter with a molecular weight cut-off of 5 kDa (Millipore). The same concentration filter is used to perform a buffer exchange to PBS. The purified IgG fraction is sterile filtered using a Millipore Millex filter (Millipore), and either snap frozen in liquid N₂ and kept at −80° C. for long term storage, or stored at 4° C. for a limited time.

Neutralizing activity of preS1-specific polyclonal IgG is done essentially as described (Glebe et al., 1993, J. Virol. 77, 9511-9521). Briefly, purified hepatitis B virus genotype D from a chronic carrier (1×10⁸ genomes per well) is preincubated with purified polyclonal IgG (0.1 to 100 μg/ml) for 1 hour at 20° C. Primary tupaia belangeri hepatocytes (5×10⁵ per well) are then incubated with the viral inoculum for 10 hours at 37° C., after which cells are washed extensively and incubation at 37° C. is continued. Medium is changed every 3 days and the amount of hepatitis B e antigen produced is determined from 9 to 12 days after infection by a commercially available enzyme-linked immunosorbent assay (AxSYM, Abbott Laboratories).

### Example 16

### Cloning, Expression, Purification and Packaging of AP205 VLP Displaying the CCR5 Peptides Cloning and Expression

Peptides corresponding to the second extracellular loop (ECL2) and to the N-terminus (Nt) of human CCR5 (with a Cys20 to Ser mutation to avoid oxidation problems) were fused to AP205 in order to generate vaccines which elicit antibodies against human CCR5. The vaccines are subsequently injected in mice, and the antibodies are tested for HIV neutralization activity. We tested, in addition to the N-terminal peptide, two loop peptides, one corresponding to the full-length of ECL2 with engineered N-terminal and C-terminal cysteines and containing a Cys to Ser mutation in position 11 to avoid interfering with loop formation, and the other one being ECL2a with an engineered cysteine at the N-terminus. Both loops therefore can be closed by disulfide bond linkage between N- and C-terminal cysteines within the loops.

The DNA fragment coding for the CCR5 peptides ECL2 with Cysteine at 11 changed to Serine (CRSQKEGLHYTSSSHFPYSQYQFWKNFQTLKIC, cECL2c, SEQ ID NO: 73), Nt with a Cys20 to Ser, (MDYQVSSPIYDINYYTSEPSQKINVKQIAAR, SEQ ID NO: 90) or ECL2a (CRSQKEGLHYTC, cECL2a, SEQ ID NO: 74) were created by annealing either two phosphorylated complementary oligodeoxynucleotides with overhangs—oligo 2-I (5′-CCGGATGTCGATCGCAGAAGGAAGGCCTACATTACACATCCTCATCTCACTTCCCA TATTCTCAATATCAATTCTGGAAGAATTTCCAAACTCTGAAGATCTGTTAATGCA-3′ SEQ ID NO: 86) and oligo 2-II (5′-TTAACAGATCTTCAGAGTTTGGAAATTCTTCCAGAATTGATATTGAGAATATGGGA AGTGAGATGAGGATGTGTAATGTAGGCCTTCCTTCTGCGATCGACAT-3′, SEQ ID NO:87) for cECL2c, oligo (CATGGATTATCAAGTCTCGAGCCCTATCTATGACATTAACTATTACACTTCGGAAC CTTCGCAGAAGATTAACGTTAAACAAATTGCAGCACGTT, SEQ ID NO: 92) and oligo (CCGGAACGTGCTGCAATTTGTTTAACGTTAATCTTCTGCGAAGGTTCCGAAGTGTA ATAGTTAATGTCATAGATAGGGCTCGAGACTTGATAATC, SEQ ID NO:93) for Nt, or two oligodeoxynucleotides—oligo 3-I (5′-GTTCCGGATGTCGATCGCAGAAGGAAGGCCTACATTACACATGCTAAT GCATGT-3′, SEQ ID NO: 88) and oligo 3-II (5′-ACATGCATTAGCATGTGTAATGTAGGCCTTCCTTCTGCGATCGACATC CGGAAC-3′, SEQ ID NO:89) for ECL2a.

The obtained fragment coding for cECL2a was additionally digested with Kpn2I and Mph11031. The three DNA fragments were subsequently ligated into the previously digested vector pAP405 (cECL2a and cECL2c) and pAP378 (Nt), respectively under the control of E. coli tryptophan operon promoter. The resulting construct were:

542: (based on 405): AP205 coat protein—GTAGGGSG—CRSQKEGLHYTSSSHFPYSQYQFWKNFQTLKIC
530: (based on 405): AP205 coat protein—GTAGGGSG—CRSQKEGLHYTC
541: (based on 378): MDYQVSSPIYDINYYTSEPSQKINVKQIAAR—SGG—AP205 coat protein

The resulting plasmids pAP542, pAP530 and pAP541 were transformed into E. coli JM109 and expressed as described in EXAMPLE 2. Capsids were identified in the lysates of all three constructs, demonstrating self-assembly of the VLP upon expression in E. coli of the respective AP205 coat protein fusion.

### Purification Construct 542

Cells were lysed by ultrasonication in lysis buffer (50 mM Tris, 5 mM EDTA 0.1% Tween 20, pH 8.0) supplemented with 5 μg/ml PMSF. The lysate was clarified by centrifugation, and the pellet washed three times with lysis buffer. The pooled supernatants were purified over a Sepharose 4B column in NET buffer. Eluted fractions containing the VLPs were pooled, concentrated using an Amicon centrifugal filter unit, and purified over a Sepharose 2B column in NET buffer. Particle assembly and display of the ECL2 peptide was demonstrated by analysis of purified VLPs by SDS-PAGE, Western Blot with a mouse anti-sera specific for the ECL2 peptide and EM.

### Purification of Construct 530

Cells were lysed by ultrasonication in lysis buffer (50 mM Tris, 5 mM EDTA 0.1% Tween 20, pH 8.0, 5 μg/ml PMSF). The lysate was clarified by centrifugation, and the pellet washed three times with lysis buffer containing 10 mM DTT. The pooled supernatants of the washes were purified over a Sepharose 4B column in NET buffer. Eluted fractions containing the VLPs were pooled, supplemented with 10 mM DTT, concentrated using an Amicon centrifugal filter unit and rechromatographed over a Sepharose 4B column in NET buffer. Particle assembly and display of the ECL2a peptide was demonstrated by analysis of purified VLPs by SDS-PAGE, Western Blot with a mouse anti-sera specific for the ECL2a peptide and EM.

In order to promote disulfide bond linkage of the two cysteines of the ECL2a peptide and hence closing of the ECL2a loop, the VLP preparation obtained above is dialyzed against 50 mM Tris, 150 mM Nacl, pH 8.0, containing 0.1 to 1 mM reduced glutathion and 0.2 to 5 mM oxidized glutathion. Subsequently, the dialyzed VLP preparation is dialyzed further against 50 mM Tris, 150 mM NaCl, pH 8.0 or PBS or 20 mM Hepes, 150 mM NaCl, pH 7.2, and is injected in mice to test immunogenicity of the displayed epitope.

### Purification of Construct Under Mild Reducing Conditions

Cells are lysed as described above in a lysis buffer containing 0.1 mM DTT. All subsequent steps are performed in buffers containing 0.1 mM DTT. Final oxidation of the internal cysteines of the ECL2a loop can optionally be performed as described above, if unsufficient disulfide bond formation is suspected.

### Example 17

### Cloning, Expression, Purification and Packaging of AP205 VLP Displaying the CXCR4 N-Terminal Peptide

### Cloning and Expression

A Peptide corresponding to the N-terminus of human CXCR4 (with a Cys28 to Ser mutation to avoid oxidation problems) was fused to AP205 in order to generate a vaccine which elicit antibodies against human CXCR4. The vaccine is subsequently injected in mice, and the antibodies tested for HIV neutralization activity. The DNA fragment coding for the CXCR4 N-terminal peptide (CXCR4-Nt) (MEGISIYTSDNYTEEMGSGDYDSMKEPSFREENANFNKI, SEQ ID NO: 75), was created by annealing two 5′ phosphorylated oligonucleotides—oligo Oligo 4-I (5′-CATGGAAGGAATTTCCATATATACTTCGGACAACTACACCGAGGAAATGGGTAGC GGCGACTACGACAGCATGAAAGAACCATCCTTCCGCGAGGAGAATGCAAATTTTA ATAAAATTT-3′, SEQ ID NO: 76) and oligo Oligo 4-II (5′-CCGGAAATTTTATTAAAATTTGCATTCTCCTCGCGGAAGGATGGTTCTTTCATGCTG TCGTAGTCGCCGCTACCCATTTCCTCGGTGTAGTTGTCCGAAGTATATATGGAAATT CCTTC-3′, SEQ ID NO:77). The obtained fragment was ligated in the vector pAP378 previously digested with NcoI and Kpn2I. The resulting construct was:

543: (based on 378):

MEGISIYTSDNYTEEMGSGDYDSMKEPSFREENANFNKI—SGG—AP205 coat protein

The resulting plasmids pAP543, was transformed into E. coli JM109 and expressed as described under Example 2. Capsids present in the lysate demonstrated self-assembly of the VLP upon expression in E. coli of the respective AP205 coat protein fusion.

### Purification

Cells were lysed by ultrasonication in lysis buffer (50 mM Tris, 5 mM EDTA 0.1% Tween 20, pH 8.0) supplemented with 5 μg/ml PMSF. The lysate was clarified by centrifugation, and the pellet washed with lysis buffer containing 1 M urea. The pooled supernatants were purified over a Sepharose 4B column in NET buffer. Eluted fractions containing the VLPs were pooled, concentrated using an Amicon centrifugal filter unit, and purified over a Sepharose 6B column in NET buffer. Particle assembly and display of the CXCR4-Nt peptide was demonstrated by analysis of purified VLPs by SDS-PAGE, Western Blot with a mouse anti-sera specific for the CXCR4-Nt peptide and EM.

### Example 19

### Immunisation and HIV-Neutralisation Assay

C57BL/6 mice were primed with 50 μg Nt-AP205, AP205-cECL2c, AP205-cECL2A, CXCR4-Nt-AP205 VLPs obtained from EXAMPLE 17 and 18 on day 0, (subcutaneously, in 0.2 ml PBS) and compared to BalbC mice primed with 50 μg construct 378 and 405 VLPs, respectively. After boosting with the same vaccines on day 14, the α-AP205 and the α-CCR5, α-CXCR4 antibody titers are checked by ELISA at day 14 and day 21.

### Purification of Polyclonal Mouse IgG

Serum immunised mice is centrifuged for five minutes at 14'000 rpm. The supernatant is loaded on a column of 3.3 ml prewashed protein G sepharose (Amersham Biosciences, Otelfingen, Switzerland). The column is then washed with PBS and eluted with 100 mM glycine pH2.8. 1 ml fractions are collected in tubes previously provided with 120 μl 1 M Tris pH8. Peak fractions absorbing at 280 nm are pooled.

### FACS Staining of Cellular CCR5 with Polyclonal Mouse IgG

CEM.NKR-CCR5 is a CCR5-expressing variant of the CEM.NKR cell line, a human line that naturally expresses CD4 (Trkola et al., J. Virol., 1999, page 8966). CEM.NKR-CCR5 cells are grown in RPMI 1640 culture medium (with 10% FCS, glutamine, and antibiotics). Cells are pelleted and resuspended in phosphate-buffered saline (PBS) containing 1% fetal calf serum (FCS) in order to get 2.3×10⁶ cells/ml. 2 mg/l rat-α-mouse-CD16/CD32 (Fcγ) (Pharmingen, Basel, Switzerland) are added as a blocking agent and incubated for 20 minutes. The cells are washed once in 1% FCS/PBS and 0.1 ml (2.3×10⁵ cells/well) are plated and then pelleted in a V-bottom 96-well plate. The cells are then resuspended with 0.1 ml α-CCR5 polyclonal antibodies (350 mg/l, 35 mg/l, 3.5 mg/l or 0.35 mg/l; eluted from protein G column; dilutions with 1% FCS/PBS). After 30 minutes at 4° C., the cells are washed once in 1% FCS/PBS and stained for 20 minutes at 4° C. with 15 mg/l FITC-goat-α-mouse-IgG (Jackson, Milan Analytica, LaRoche) in 1% FCS/PBS. After two washes in 1% FCS/PBS, 5,000-10,000 stained cells are analysed by flow cytometry. The geometric mean of each staining is determined using the “cell quest” flow cytometry software.

### HIV-Neutralisation Assay

### Stimulated Primary CD8 Depleted PBMC

Briefly, buffy coats obtained from 3 healthy blood donors are depleted of CD8+ T cells using Rosette Sep cocktail (StemCell Technologies Inc., BIOCOBA AG) and PBMC isolated by Ficoll-Hypaque centrifugation (Amersham-Pharmacia Biotech). Cells are adjusted to 4×10⁶/ml in culture medium (RPMI 1640, 10% FCS, 100 U/ml IL-2, glutamine and antibiotics), divided into three parts and stimulated with either 5 μg/ml phytohemagglutinin (PHA), 0.5 μg/ml PHA or 1 mg/l anti-CD3 MAb OKT3. After 72 h, cells from all three stimulations are combined and used as source of stimulated CD4+ T cells for infection and virus neutralisation experiments.

HIV neutralisation assay is performed essentially as described previously (Trkola et al., J. Virol., 1999, page 8966). The R5 viruses (CCR5 co-receptor specific strains), JR-FL and SF162, have been described previously (O'Brien et al., Nature 1990, 348, page 69; and Shioda et al., Nature 1991, 349, page 167). Alternatively, the X4 strains NL4-3 and 2044 have been described previously (Trkola et al (1998), J. Virol. 72:396; Trkoly et al (1998), J. Virol 72-1876). Briefly, cells are incubated with serial dilutions of purified polyclonal mouse IgG or control antibody 2D7 (25 μg/ml-25 ng/ml; Pharmingen) in 96-well culture plates for 1 h at 37° C.

The HIV-1 inoculums are adjusted to contain approximately 1,000 to 4,000 TCID₅₀/ml in assay medium (TCID₅₀: 50% tissue culture infective dose, Trkola et al., J. Virol., 1999, page 8966). Virus inoculum (100 TCID₅₀; 50% tissue culture infective dose;) is added and plates cultured for 4-14 days. The total infection volume is 200 μl. Preferably, on day 6 post infection, the supernatant medium is assayed for the HIV-1 p24 antigen production by using an immunoassay, as described previously (Moore et al., 1990. Science 250, page 139).

### Example 19

### Cloning, Expression, Purification and Packaging of AP205 VLP Displaying the P33 Epitope at the C-Terminus of its Coat Protein

### Cloning and Expression

The DNA fragment coding for the P33 peptide modified with a Leucine added at the N-terminal for improved processing in antigen presenting cells (LKAVYNFATM, SEQ ID NO: 78) was created by annealing two oligonucleotides—oligo 2.198 (5′-CCTCCGGACTGAAA GCTGTGTATAACTTCGCGACTATGTAATGCATCG-3′, SEQ ID NO: 79) and oligo 2.199 (5′-CGATGCATTACATAGTCGCGAAGTTATACACAGCTTTCAGTCCGGAGG-3′, SEQ ID NO:80). The obtained fragment was digested with Kpn2I and Mph1103I and cloned in the same restriction sites into the vector pAP409 under the control of E. coli tryptophan operon promoter. The resulting construct was:

425: (based on 409): AP205 coat protein—GSG—LKAVYNFATM

The resulting plasmid was named pAP425 and was transformed into E. coli JM109 and expressed as described under Example 2.

### Purification

Cells were lysed by ultrasonication in lysis buffer (50 mM Tris, 5 mM EDTA 0.1% Triton X100, pH 8.0) supplemented with 5 μg/ml PMSF. The lysate was clarified by centrifugation, and the pellet washed twice with lysis buffer and once with lysis buffer containing 1 M urea. The pooled supernatants were purified over a Sepharose CL-4B column in NET buffer. Eluted fractions containing the VLPs were pooled, concentrated using an Amicon centrifugal filter unit, and purified over a CL-6B column in NET buffer. Particle assembly and display of the P33 peptide was demonstrated by analysis of purified VLPs by SDS-PAGE, Western Blot with a mouse anti-sera specific for the P33 peptide and EM. The yield of protein was 2.7 mg/g cells. The P33 peptide can therefore successfully be fused to the C-terminus of AP205 coat protein leading to abundant particle formation. The present result show that the modified AP205 VLPs are a robust system for the fusion of epitopes such as P33, which when fused to another RNA phage VLP such as Fr prevent particle assembly.

### Packaging

AP205-p33 obtained above (2.7 mg/ml), was dialyzed against 20 mM Hepes, pH 7.4, and digested with RNAse A (300 μg/ml VLP) at 37° C. for 3 hrs. The RNAse treated VLP was subsequently dialyzed overnight at 4° C. (Molecular weight cutoff=100000). Oligodeoxynucleotides (oligos) 1668 pt (t*c*c*a*t*g*a*c*g*t*t*c*c*t*g*a*a*t*a*a*t, where * means phosphorothioate bond, SEQ ID NO:94) and NKpt (g*g*g*g*t*c*a*a*c*g*t*t*g*a*g*g*g*g*g*g, SEQ ID NO:95) were packaged in AP205-p33 as follows. 0.12 ml of a 1 mM oligo stock/ml treated VLP and MgCl₂ (final 2 mM) were added and incubated for 3 hours at 37° C. Free oligo was removed by tangential flow filtration using a 20 mM Hepes, pH 7.4 buffer. Packaging of oligo was confirmed by analysis of the reassembled VLPs by agarose gel electrophoresis in ethidium bromide. Residual free oligo in the packaged VLP preparation was quantified by comparison with 8 dilutions of a standard of the same oligo on the same gel. The total amount of oligo was quantified by treating the packaged VLP preparation with proteinase K and analysis by PAGE on 10% TBE/urea gels. The gels were stained with SYBR gold, and total oligo content of each band quantified by densitometry using 5 dilutions of the same oligo as standard. Residual oligo content was subtracted from the total oligo content to yield the packaged oligo content, which was of 4.36 nmol/100 μg VLP of NKpt oligo and 2.98 nmol/100 μg VLP of 1668 pt oligo.

### Example 20

### Induction of a CD8+ T Cell Response by AP205 p33 Fusion Protein Reassembled in the Presence of Oligodeoxynucleotides

C57BL/6 mice are immunized by injecting subcutaneously 150 ug of AP205-p33 (construct 425) with oligo 1668 pt or oligo NKpt packaged inside as described in EXAMPLE 19, or AP205-p33 VLPs reassembled in the presence of different amount of poly-L-glutamic acid, which is not a ligand to Toll-like receptor (AP205-p33/poly L-Glu with 0.1 mg/ml, 0.2 mg/ml or 0.4 mg/ml of poly-L-glutamic acid). Eight days later blood from immunized animals is analysed for the expansion of gp33-specific CD8+ T cells. Blood is collected in FACS buffer (PBS, 2% FCS, 5 mM EDTA, pH 8.2) and stained for 10 min at 37° C. with PE-labeled H2-Db-tetramer loaded with the gp33-peptide (Proimmune) followed by staining for 30 min at 4° C. with an APC labelled rat anti-mouse CD8a-antibody (BD PharMingen). After washing, erythrocytes are lysed with BD-Lyzing Solution (BD Biosciences, San Jose, USA) for 10 min at room temperature. Finally, the cells are analysed on a FACS Calibur using CellQuest software. First of all, the cells are acquired in the forward scatter and side scatter and the lymphocytes are gated. From this lymphocyte population, the gp33-PE labelled and CD8-APC labelled cells are measured with the FL2 and FL4 detector, respectively. The amount of gp33-specific T cells are calculated as percent CD8 positive, gp33 positive cells on total CD8 positive lymphocytes.

After the measurement of the gp33-specific T cell response the mice are challenged with 1.5×10⁶ pfu of a recombinant vaccinia virus that expresses the gp33-peptide. 5 days later the viral titer is measured in the ovaries of these mice. A single cell suspension of the ovaries is incubated in serial dilutions on BSC40 cells. After overnight incubation at 37° C. at 5% CO₂ cells are stained with crystal violet (500 ml 96% Ethanol, 5 g Crystal violet (Sigma C-3886), 8 g NaCl, 450 ml H₂O, 50 ml Formaldehyde) in order to visualize plaques in the cell layer derived from virus induced cell lysis. The number of residual virus in the ovaries is calculated as plaque forming units (pfu).

### Example 21

### Cloning, Expression, and Purification of AP205 VLP Displaying the Ghrelin Peptide

### Cloning and Expression

A peptide corresponding to human Ghrelin(1-8) was fused to the C-terminus of AP205 in order to generate a vaccine which elicit antibodies against Ghrelin. The vaccine is subsequently injected in mice, and the antibodies tested for binding to Ghrelin. The DNA fragment coding for the Ghrelin peptide (GSSFLSPE, SEQ ID NO: 55), was created by annealing two oligonucleotides—Oligo 4.173 (5′-GT TCC GGA GGG AGC TCC TTC CTG TCT CCG GAA TAA TGCATGT-3′, SEQ ID NO: 81) and Oligo 4.174 (5′-ACATGCA TTA TTC CGG AGA CAG GAA GGA GCT CCC TCC GGA AC-3′, SEQ ID NO:82). The obtained fragment was digested with Kpn2I and Mph1103I and cloned in the same restriction sites into the vector pAP405 and pAP409, under the control of E. coli tryptophan operon promoter. The resulting constructs were:

(based on 405) 513 AP205 coat protein—GTAGGGSG—GSSFLSPE
(based on 409) 514 AP205 coat protein—GSG—GSSFLSPE

The resulting plasmids pAP513 and pAP514, were transformed into E. coli JM109 and expressed as described under Example 2. Capsids present in the lysate demonstrated self-assembly of the VLP upon expression in E. coli of the respective AP205 coat protein fusion.

### Purification of VLP from Construct 513

Cells were lysed by ultrasonication in lysis buffer (50 mM Tris, 5 mM EDTA 0.1% Tween 20, pH 8.0) supplemented with 5 μg/ml PMSF. The lysate was clarified by centrifugation, and the pellet washed three times with lysis buffer. The pooled supernatants were supplemented with NaCl to a final concentration of 0.4 M, and precipitated with one half volume of a 40% PEG 6000 solution in H2O. The precipitate was isolated by centrifugation, washed and resuspended in H2O and purified over a Sepharose CL-2B column in NET buffer. Eluted fractions containing the VLPs were pooled, and concentrated using an Amicon centrifugal filter unit. Protein from construct 513 was further purified over a sucrose gradient prepared with the following sucrose solutions: 9 ml 36%, 3 ml 30%, 6 ml 25%, 8 ml 20%, 6 ml 15%, 6 ml 10% and 3 ml 5%. The VLP fractions were pooled, concentrated over a centrifugal filter unit and dialyzed against 10 mM Hepes, pH 7.5. Concentrated fractions containing VLPs from the CL-2B purification run of protein from construct 514 were further purified over a Sepharose 6B column, and fractions containing VLPs were concentrated over a centrifugal filter unit.

Display of the Ghrelin peptides on AP205 particles was demonstrated by analysis of the purified VLPs by SDS-PAGE, Western blot with a mouse antiserum specific for Ghrelin, inhibition ELISA inhibiting the binding of the mouse serum specific for Ghrelin to the Ghrelin peptide conjugated to RNAse and coated on an ELISA plate with AP205-Ghrelin VLPs, and EM.

Adult female, C57BL/6 mice (5 per group) are vaccinated with purified VLP from construct 513, purified VLP from construct 405 is used as negative control. Alternatively adult female, C57BL/6 mice (5 per group) are vaccinated purified VLP from construct 514, purified VLP from construct 409 is used as negative control. 100 μg of dialyzed vaccine from each sample were diluted in PBS to a volume of 200 μl and injected subcutaneously (100 μl on two ventral sides) on days 0, 14, 28 and 42. Mice are bled retro-orbitally on day 0, 14, 28, 42 and 56 and their sera analyzed by ELISA.

Mice are subsequently boosted if ghrelin-specific antibody titers significantly decline during the experiment. All mice are placed on a high fat diet (35% fat by weight, 60% as energy) to facilitate the development of diet-induced obesity. Food and water is administered ad libitum. Body weights are monitored at regular intervals.

### Example 22

### Cloning, Expression, Purification and Packaging of AP205 VLP Displaying the M2 Peptide at its N-Terminus

### Cloning and Expression

A peptide corresponding to an M2 peptide from Influenza virus was fused to the N-terminus of AP205 in order to generate a vaccine which elicit antibodies against Influenza protein M2. The vaccine is subsequently injected in mice, and the protective effect of the immunization assessed. The DNA fragment coding for the M2 peptide with MG added at the N-terminus (MGSLLTEVETPIRNEWGCRCNDSSDG, SEQ ID NO: 83), was created by annealing two 5′ phosphorylated oligodeoxynucleotides—oligo M2-I (5′-GGC CAT GGG ATC TCT GCT GAC CGA AGT TGA AAC CCC GAT TCG TAA TGA ATG GGG TTG CCG TTG CAA TGA TTC TTC TGA TGG TTC CGG AGG-3′, SEQ ID NO: 84) and oligo M2-II (5′-CCT CCG GAA CCA TCA GAA GAA TCA TTG CAA CGG CAA CCC CAT TCA TTA CGA ATC GGG GTT TCA ACT TCG GTC AGC AGA GAT CCC ATG GCC-3′, SEQ ID NO:85). The obtained fragment was cloned in the vector pAP378 previously digested with NcoI and Kpn2L. The resulting construct was:

551: (based on 378): MGSLLTEVETPIRNEWGCRCNDSSDG—SGG—AP205 coat protein

The resulting plasmids pAP551, was transformed into E. coli JM109 and expressed as described in EXAMPLE 2. Capsids present in the lysate demonstrated self-assembly of the VLP upon expression in E. coli of the AP205 coat protein fusion.

### Purification

Cells were lysed by ultrasonication in lysis buffer (50 mM Tris, 5 mM EDTA 0.1% Tween 20, pH 8.0) supplemented with 5 μg/ml PMSF. The lysate was clarified by centrifugation, and the pellet washed with lysis buffer containing 1 M urea. The pooled supernatants were purified over a Sepharose CL-4B column in NET buffer. Eluted fractions containing the VLPs were pooled, concentrated using an Amicon centrifugal filter unit, and purified over a Sepharose 6B column in NET buffer. The fractions containing VLPs were pooled, concentrated with a centrifugal filter unit and dialyzed against 10 mM Hepes, pH 7.5, Particle assembly and display of the M2 peptide was demonstrated by analysis of purified VLPs by SDS-PAGE and EM.

### Example 23

### Cloning, Expression, Purification AP205 VLP Displaying the M2 Peptide at its C-Terminus

### Cloning and Expression

A peptide corresponding to an M2 peptide from Influenza virus is fused to the C-terminus of AP205 coat protein. Briefly, two complementary oligonucleotides encoding the M2 sequence (SEQ ID NO:43) to be fused in frame with the AP205 coat protein, flanked by Kpn 2I and Mph 11031 restriction sites for cloning are synthesized. A stop codon is also included at the end of the peptide coding sequence. The complementary oligonucleotides are annealed, digested with Kpn 2I and Mph 11031 and cloned into pAP409, pAP405 to generate C-terminal fusions. The resulting constructs are:

| | |
|---|---|
| pAP409-M2: | AP205-GSG-SLLTEVETPIRNEWGCRCNDSSDG |
| pAP405-M2: | AP205-GTAGGGSG-SLLTEVETPIRNEWGCRCNDSSDG |

The corresponding fusion proteins are expressed and purified substantially as described in EXAMPLE 22.

### Example 24

### Cloning, Expression and Purification of AP205 VLPs Displaying Multimers of the M2 Peptide Fused Either to the C- or N-Terminus

### Cloning of Expression Vectors

Multimers of the M2 peptide in tandem separated with short spacer sequences are in-frame fused to the AP205 coat protein. Briefly, two complementary oligonucleotides encoding the desired sequence (see below) to be fused in frame with the AP205 coat protein, flanked by appropriate restriction sites for cloning are synthesized. A stop codon is also included at the end of the peptide coding sequence for fusions to the C-terminus of AP205 (cloning into pAP409, pAP405). The complementary oligonucleotides are annealed, digested with the appropriate restriction enzymes and cloned into the respective AP205 fusion vector. M2 dimer (SLLTEVETPIRNEWGCRCNDSSDG-GSSG-SLLTEVETPIRNEWGCRCNDSSDG, SEQ ID NO:96) or an M2 trimer (SLLTEVETPIRNEWGCRCNDSSDG-GSSG-SLLTEVETPIRNEWGCRCNDSSDG-GSSG-SLLTEVETPIRNEWGCRCNDSSDG, SEQ ID NO:97) are cloned into pAP378 and pAP382 to generate N-terminal fusions and into pAP409 and pAP405 to generate C-terminal fusions. The corresponding fusion proteins are expressed and purified substantially as described in EXAMPLE 22.

### Example 25

### Functional Testing of AP205 VLPs Displaying the M2 Peptide or Multimers Thereof on the Surface

In order to test the different AP205-M2 fusion vaccines mice are immunised with the VLPs obtained from EXAMPLES 22-24 and subsequently subjected to Influenza A virus challenge essentially as previously described (Jegerlehner et al., J. Immunol., 2004, page 5598-5605). Briefly, adult C57BL/6 mice (5 per group) are vaccinated with the VLPs obtained from EXAMPLES 22-24 respectively and with AP205 VLP as negative control. For each mouse, 100 μg of vaccine is diluted in PBS to a volume of 2001 and injected subcutaneously into the right and the left inguinal region of each animal on days 0 and 14. Animals are bled on day 14 and 21, and the M2 specific antibody response is measured in an ELISA. Briefly, M2 peptide is conjugated to RNase via an amino acid spacer (CGG) and the cross-linker SPDP and coated on an ELISA plate overnight at 4° C. Binding of the sera is detected with a Horseradish-peroxidase goat anti-mouse IgG conjugate.

On day 33 all mice are challenged with 4000 live Influenza A viruses (strain: A/Puerto Rico 8/34, H1N1 subtype)/mouse. The body weight and mortality in each vaccinated group is then monitored over 14 days.

### Example 26

### Cloning, Expression and Purification of Modified VLP Comprising Fusion Proteins of the AP205 Coat Protein with HIV env Peptides

### Cloning and Expression

Peptides (SEQ ID NOs:98-113) derived from HIV envelope glycoprotein gp 160 are in-frame fused to the AP205 coat protein. Briefly, two complementary oligonucleotides encoding the desired sequence to be fused in frame with the AP205 coat protein, flanked by appropriate restriction sites for cloning are synthesized. A stop codon is also included at the end of the peptide coding sequence for fusions to the C-terminus of AP205 (cloning into pAP409, pAP405). A initial Methione codon is added at the beginning of the Oligos for fusing at the N-terminus of AP205. The complementary oligonucleotides are annealed, digested with the appropriate restriction enzymes and cloned into the respective AP205 fusion vector. The HIV env peptides are cloned into pAP378 and pAP382 to generate N-terminal fusions and into pAP409 and pAP405 to generate C-terminal fusions.

The expression and purification of the corresponding fusion proteins are substantially carried out as described in EXAMPLE 2 and 3.

Adult C57BL/6 mice (5 per group) are vaccinated with the AP205-HIV env peptides, respectively, as a control, with AP205 VLP. For each mouse, 100 μg of vaccine is diluted in PBS to a volume of 2001 and injected subcutaneously into the right and the left inguinal region of each animal on days 0 and 14.

Animals are bled on day 14 and 21, and the antibody response is measured in an ELISA. Briefly, the antigen to-be-tested is conjugated to RNase via an amino acid spacer (CGG) and the cross-linker SPDP and coated on an ELISA plate overnight at 4° C. Binding of the sera is detected with a Horseradish-peroxidase goat anti-mouse IgG conjugate.

Mice are then bled out by heart puncture on day 21 and serum of each vaccine is purified as follows: sera from the 5 mice of the respective group are pooled and centrifuged for five minutes at 14,000 rpm. The supernatant is loaded on a column of 3 ml prewashed protein G sepharose (Amersham Biosciences). The column is then washed with 10 column volumes of PBS and eluted with 100 mM glycine pH2.8. 1 ml fractions are collected in tubes containing 200 μl 1M Tris pH8.0. The protein containing fractions are pooled and concentrated using a Millipore Ultrafree centrifugal filter with a molecular weight cut-off of 5 kDa (Millipore). The same concentration filter is used to perform a buffer exchange to PBS. The purified IgG fraction is sterile filtered using a Millipore Millex filter (Millipore), and either snap frozen in liquid N₂ and kept at −80° C. for long term storage, or stored at 4° C. for a limited time.

The obtained sera are then tested in HIV neutralisation assays as essentially described in EXAMPLE 19 with R5, X4 virus strains. Moreover purified IgG fractions are tested for their ability to neutralise primary HIV isolates as described previously (Hovanessian et al., Immunity 2004, page 617-627).

1. A modified virus-like particle (VLP) comprising at least one fusion protein, wherein said at least one fusion protein comprises:
(a) a first polypeptide; and
(b) a second polypeptide,
wherein said first polypeptide is a coat protein, or a mutein thereof, of AP205 bacteriophage, and wherein said second polypeptide is fused to said first polypeptide either to the N- or to the C-terminus of said first polypeptide.

2. The modified VLP of claim 1, wherein said second polypeptide comprises at least one antigen.

3. The modified VLP of claim 1, wherein said first polypeptide consists of 124-138 amino acids.

4. The modified VLP of claim 1, wherein said coat protein, or a mutein thereof, of AP205 bacteriophage is selected from a group consisting of:
(a) SEQ ID NO:1;
(b) SEQ ID NO:2;
(c) SEQ ID NO:42;
(d) SEQ ID NO:67;
(e) SEQ ID NO:68;
(f) SEQ ID NO:69; and
(g) a mutein of SEQ ID NO:1 or 67.

5. The modified VLP of claim 4, wherein said mutein has the amino acid sequence as set forth in SEQ ID NO:1 or 67; and wherein at most three amino acid residues of SEQ ID NO:1 or 67 are deleted, internally added, or substituted.

6. The modified VLP of claim 1, wherein said fusion protein further comprises a spacer, and wherein said spacer is positioned between said first polypeptide and said second polypeptide.

7. The modified VLP of claim 6, wherein said spacer has at most 15 amino acids.

8. The modified VLP of claim 1, wherein said second polypeptide comprises at least one antigen selected from a group consisting of:
(a) an antigen suited to induce an immune response against cancer cells;
(b) an antigen suited to induce an immune response against at least one microbial pathogen;
(c) an antigen suited to induce an immune response against at least one allergen;
(d) an antigen suited to induce an immune response against at least one self antigen;
(e) an antigen suited to induce an immune response in farm animals or pets; and
(f) an antigen suited to induce a response against a polypeptide toxin or a polypeptide hormone.

9. The modified VLP of claim 1, wherein said at least one antigen is selected from the group consisting of:
(a) a polypeptide of HIV;
(b) a polypeptide of Hepatitis B virus;
(c) a polypeptide of Influenza virus;
(d) a polypeptide of Hepatitis C virus;
(e) a polypeptide of Toxoplasma;
(f) a polypeptide of Plasmodium falciparum;
(g) a polypeptide of Plasmodium vivax;
(h) a polypeptide of Plasmodium ovale;
(i) a polypeptide of Plasmodium malariae;
(j) a polypeptide of Chlamydia;
(k) a polypeptide of breast cancer cells,
(l) a polypeptide of kidney cancer cells,
(m) a polypeptide of prostate cancer cells,
(n) a polypeptide of skin cancer cells,
(o) a polypeptide of brain cancer cells,
(p) a polypeptide of leukemia cells,
(q) a recombinant profiling,
(r) a polypeptide involved in bee sting allergy,
(s) a polypeptide involved in nut allergy,
(t) a polypeptide involved in food allergies,
(u) a polypeptide involved in asthma,
(v) Her2;
(w) GD2;
(x) EGF-R;
(y) CEA;
(z) CD52;
(aa) Human melanoma gp100;
(bb) Human melanoma melanA
(cc) Tyrosinase;
(dd) NA17-A nt;
(ee) MAGE3;
(ff) P53;
(gg) CD21;
(hh) HPV16E7;
(ii) a phospholipase A₂ protein;
(jj) a Der p I peptide;
(kk) an Influenza M2 protein;
(ll) a fragment of said at least one antigen of (a) to (z) and of (aa) to (kk); and
(mm) a variant of said at least one antigen of (a) to (z) and of (aa) to (kk).

10. The modified VLP of claim 1, wherein said at least one antigen is a self antigen.

11. The modified VLP of claim 10, wherein said self antigen is a polypeptide, selected from the group consisting of:
(a) lymphotoxins (preferably Lymphotoxin α (LT α), Lymphotoxin β (LT β));
(b) lymphotoxin receptors;
(c) receptor activator of nuclear factor kB ligand (RANKL);
(d) vascular endothelial growth factor (VEGF);
(e) vascular endothelial growth factor receptor (VEGF-R);
(f) Interleukin-5;
(g) Interleukin-17;
(h) Interleukin-13;
(i) IL-23 p19;
(j) Ghrelin;
(k) CCL21;
(l) CXCL12;
(m) SDF-1;
(n) M-CSF;
(o) MCP-1;
(p) Endoglin;
(q) GnRH;
(r) TRH;
(s) Eotaxin;
(t) Bradykinin;
(u) BLC;
(v) Tumor Necrosis Factor α;
(w) amyloid beta peptide (Aβ₁₋₄₂);
(x) Aβ_{1-6;}
(y) Angiotensin;
(z) CCR5 extracellular domain;
(aa) CXCR4 extracellular domain;
(bb) Gastrin;
(cc) CETP;
(dd) C5a;
(ee) Bradykinin;
(ff) Des-Arg Bradykinin
(gg) a fragment of (a)-(ff); and
(hh) a variant of (a)-(ff).

12. The modified VLP of claim 1, wherein said second polypeptide consists of 5-30 amino acids.

13. The modified VLP of claim 1, wherein said second polypeptide comprising an amino acid sequence selected from the group consisting of:
(a) Influenza virus M2 peptide (SEQ ID NO:43);
(b) Hepatitis B virus Pre S1 peptide (SEQ ID NO:62);
(c) HIV Nef Polyepitops (SEQ ID NO:23);
(d) GnRH (SEQ ID NO:20);
(e) Gastrin G17 (SEQ ID NO:47);
(f) Cat Ghrelin (SEQ ID NO:59);
(g) Dog Ghrelin (SEQ ID NO:58);
(h) HIV Env peptide 1 (SEQ ID NO:98);
(i) HIV Env peptide 2 (SEQ ID NO:99);
(j) CCR5 PNt (SEQ ID NO:45); and
(k) CCR5 ECL2 (SEQ ID NO:91).

14. The modified VLP of claim 1 further comprising at least one immunostimulatory nucleic acid, wherein said immunostimulatory nucleic acid is packaged inside said modified VLP.

15. The modified VLP of claim 14, wherein said nucleic acid comprising at least one unmethylated CpG motif comprises the sequence GGGGGGGGGGGACGATCGTCGGGGGGGGGG (SEQ ID NO: 71).

16. A pharmaceutical composition comprising:
(a) the modified VLP of claim 1; and
(b) an acceptable pharmaceutical carrier.

17. A vaccine composition comprising an immunologically effective amount of the modified VLP of claim 1.

18. The vaccine composition of claim 17 further comprising an adjuvant.

19. A method of immunization comprising administering the vaccine composition of claim 17 to an animal or a human.

20. A fusion protein comprising a polypeptide, wherein said polypeptide is fused to either the N- or C-terminus, or to both terminus, of a coat protein, or a mutein thereof, of AP205 bacteriophage; and wherein said polypeptide consists of 3-10 amino acids; and wherein said fusion protein is capable of forming a VLP.

21. A nucleotide sequence encoding said fusion protein of claim 20.

22. A method for producing the modified VLP of claim 1 comprising the steps of:
(a) optionally in-frame ligating a nucleotide sequence encoding a spacer with either the first nucleotide sequence encoding the first polypeptide or the second nucleotide sequence encoding the second polypeptide;
(b) in-frame ligating said second nucleotide sequence with said first nucleotide sequence, resulting in a third nucleotide sequence encoding said fusion protein;
(c) optionally introducing a stop codon which allows suppression at the 3′ of the first nucleotide sequence;
(d) expressing said third nucleotide sequence in a host, preferably under the condition that the resulting expressed proteins are capable of forming said modified VLPs;
(e) purifying said modified VLPs obtained from step (d).

23. A method of treating or preventing a disease, a disorder or physiologic conditions in an individual, wherein said method comprises administering to an animal or a human a modified VLP of claim 1 or the pharmaceutical composition of claim 16 or the vaccine composition of the claim 17.
